(19) United States
(12) Patent Application Publication
(10) Pub. No.: US 2026/0345538 A1
(43) Pub. Date: Nov. 5, 2026

(54) BETA-ALANINE COMPOSITIONS AND METHODS OF USE THEREOF

(71) Applicant: Natural Alternatives International, Inc., Carlsbad, CA (US)

(72) Inventors: Sreeraj Gopi, Chalakudy (IN); Di Tan, Valley Center, CA (US); Guru Ramanathan, Gibsonia, PA (US)

(21) Appl. No.: 19/738,168

(22) Filed: Jul. 10, 2026

Related U.S. Application Data
(63) Continuation-in-part of application No. 18/916,596, filed on Oct. 15, 2024.
(60) Provisional application No. 63/548,507, filed on Nov. 14, 2023.
(60) Provisional application No. 63/841,804, filed on Jul. 10, 2025.

(51) Int. Cl. A61K 31/197; A61K 9/16; A61K 31/015; A61K 31/522

(52) U.S. Cl. CPC A61K 31/197; A61K 9/1617; A61K 9/1652; A61K 31/015; A61K 31/522

(57) ABSTRACT

Disclosed herein are novel beta-alanine compositions for increasing athletic performance, improving cognitive function, and attenuating the aging process. The compositions described herein comprise improved bioavailability and reduced paresthesia effects compared to free beta-alanine. Compositions described herein can be administered for sports nutrition to adults (young or old) to enhance exercise performance by providing adequate carnosine-concentration increases without causing paresthesia or with reduced paresthesia. Compositions described herein can also be administered to subjects, such as elderly subject, in functional or medical foods, along with other formulations to aid in healthy aging including reduced muscle wasting, improved mobility, improved cognitive performance, and the like. Compositions described herein can be formulated as read-to-drink beverages. Compositions described herein can be mixed with foods and beverages suitable for human consumption. Also described herein are methods of making the novel beta-alanine compositions disclosed herein.

### CROSS-REFERENCE TO RELATED APPLICATIONS

This application claims the benefit of U.S. Provisional Application No. 63/841,804 filed Jul. 10, 2025 and is a continuation-in-part application of U.S. application Ser. No. 18/916,596 filed Oct. 15, 2024, which claims the benefit of U.S. Provisional Application No. 63/548,507 filed Nov. 14, 2023. The contents of which are herein incorporated by reference in its entirety.

### BACKGROUND

Carnosine can maintain the acid-base equilibrium in several forms of tissue and is synthesized from beta-alanine via the ATP-Grasp Domain-containing protein 1, which is expressed in skeletal muscle, brain, and cardiac muscle. While ingested carnosine cannot enter cells to a significant extent, beta-alanine can enter into cells and be taken up by tissues expressing ATP-Grasp Domain-containing protein 1 enzyme. As such, supplementation of beta-alanine can decrease muscle fatigue and enhance exercise performance by increasing carnosine concentrations in skeletal muscle, which in turn reduces/clears lactate. In addition, supplementation of beta-alanine can increase carnosine concentrations in heart muscle, brain, and cells. Increased carnosine concentrations in brain can reduce brain inflammation and have exhibited various cognitive benefits, including improved expression of brain-derived neurotrophic factor in the hippocampus under stress, reduced inflammatory response to stress via reduced glial fibrillary acidic protein levels, and maintenance of dendritic complexity, which leads to healthy brain function and healthy mood balance (such as better cognitive function and reduced anxiety). Moreover, carnosine can help macrophages eliminate senescent skin cells by increasing their phagocytic capacity, can positively regulate macrophage and microglial functions, has anti-inflammatory properties, and is an antioxidant, a chelator of toxic metal ions, an anti-glycating agent, and an aldehyde/carbonyl binder and thus, increased carnosine concentrations in muscle (including heart muscle) and cells can promote and maintain heart health, muscle health, bone health, and a healthy immune system, have anti-aging effects, and support general well-being including maintaining a healthy level of muscle mass and a healthy level of physical performance. Furthermore, starting as early as age 30, carnosine levels decline and the rate of decline accelerates over time during aging. Restoring carnosine levels can slow down the aging process, promote healthy aging, and support general wellness. Accordingly, improving the bioavailability of beta-alanine is believed to lead to improvements in these effects.

Declined cognitive function is common among the elderly. Increasing age is often accompanied by a progressive decline in most physiological functions such as brain, heart, bone, joint, and muscle functions and a weakened immune system. Reducing the effects of lactate buildup and fatigue can also improve physical endurance, physical performance, athletic performance, and mental acuity.

Physical exercise is vital to human health. Regular physical activity can improve muscle strength, boost endurance, and reduce the risk of developing dementia, among other diseases. However, exercise may lead to muscle fatigue due to lactate building up in skeletal muscle and muscle inflammation. Intensive exercise can also lead to declined cognitive function, which is associated with increased oxidative stress and neuroinflammation in brain. Beta-alanine supplementation can increase muscle carnosine concentrations, but prior art compositions have either led to undesirable feelings of paresthesia or lacked adequate carnosine-concentration increases.

A historical problem with the administration of beta-alanine is the sensation often experienced by users referred to as paresthesia. Paresthesia was known to present as a tingling sensation that was experienced by some users at higher doses, which were administered for beta-alanine's carnosine-boosting effects.

Therefore, to fully utilize the benefits of beta-alanine and the corresponding cellular production of carnosine, there is a need to develop improved beta-alanine compositions with increased bioavailability that enhance exercise performance, promote and maintain brain health and mood (e.g., improve cognitive function and reduce anxiety), maintain and promote heart health, muscle health, bone health, and a healthy immune system, support general well-being, and attenuate the aging process.

### SUMMARY

Embodiments of the present disclosure are based on novel beta-alanine compositions having unexpectedly increased bioavailability leading to increased cellular carnosine concentration and as a result, improved cognitive function, reduced anxiety, and improved anti-aging effects (including maintaining a healthy level of muscle mass, a healthy level of physical performance, healthy heart, healthy blood sugar levels, healthy immune system, and healthy bones and joints) improved athletic performance (including a reduction in the onset of muscle fatigue and improved endurance and working capacity). In certain embodiments, novel beta-alanine-containing compositions unexpectedly exhibited significantly reduced paresthesia effect or no paresthesia effect.

Described herein are novel beta-alanine compositions, methods of making the compositions, and methods of using the compositions.

Compositions described herein can be administered for sports nutrition to adults (young or old as described herein) to enhance exercise and physical performance by providing adequate carnosine-concentration increases without causing paresthesia or with reduced paresthesia.

These and other features, aspects, and advantages of the present embodiments will become understood with reference to the following description and appended claims.

### BRIEF DESCRIPTION OF THE DRAWINGS

FIGS. 1A, 1B, 1C, and 1D show respectively cytotoxicity of Reference product 1, Reference product 2, Composition 1 and Composition 2 described herein each at five concentrations as compared to a control composition.

FIG. 2A shows cell permeability beta-alanine from Composition 1 and Composition 2 as powders, as described herein, as compared to Reference product 1 and Reference product 2 also as powders.

FIG. 2B shows cell permeability of beta-alanine from Composition 1 and Composition 2 in saline solutions, as described herein, as compared to Reference product 1 and Reference product 2 also in saline solutions.

FIG. 2C shows cell permeability of beta-alanine from Composition 1 and Composition 2 in protein shots, as described herein, as compared to Reference product 1 and Reference product 2 also in protein shots.

### DETAILED DESCRIPTION

Described herein are novel compositions comprising one or more of beta-alanine, an anti-inflammatory agent, an amphiphilic agent, and beta-cyclodextrin. As used herein, beta-alanine can refer to free beta-alanine, an ester of free beta-alanine, a salt of free beta-alanine, or a combination thereof. Free beta-alanine can refer to beta-alanine having the chemical name of 3-amino propanoic acid, which is free of chemical modifications, is unencapsulated, and is not beta-alanine derivatives. Examples of beta-alanine esters include, but are not limited to beta-alanine methyl ester, beta-alanine ether ester, beta-alanine propyl ester, beta-alanine isopropyl ester, beta-alanine butyl ester, beta-alanine tert-butyl ester, and the like. Examples of the beta-alanine salts include, but are not limited to beta-alanine sulfate, beta-alanine citrate, beta-alanine maleate, beta-alanine acetate, beta-alanine oxalate, beta-alanine hydrochloride, beta-alanine hydrobromide, beta-alanine hydroiodide, beta-alanine nitrate, beta-alanine bisulfate, beta-alanine phosphate, beta-alanine isonicotinate, beta-alanine lactate, beta-alanine salicylate, beta-alanine tartrate, beta-alanine oleate, beta-alanine tannate, beta-alanine pantothenate, beta-alanine bitartrate, beta-alanine ascorbate, beta-alanine succinate, beta-alanine gentisate, beta-alanine fumarate, beta-alanine gluconate, beta-alanine glucoronate, beta-alanine glycinate, beta-alanine saccharate, beta-alanine formate, beta-alanine benzoate, beta-alanine glutamate, beta-alanine methanesulfonate, beta-alanine ethanesulfonate, beta-alanine benzenesulfonate, beta-alanine p-toluenesulfonate, and beta-alanine pamoate (i.e., 1,1′-methylene-bis-(2-hydroxy-3-naphthoate)), and the like.

In some embodiments, beta-alanine can be micronized prior to incorporation into the composition so that the composition has an improved particle size distribution over prior art beta-alanine compositions.

An anti-inflammatory agent, as described herein, may refer to an agent capable of reducing inflammation. Without being bound by any particular theory, it is believed that an anti-inflammatory agent can aid in reducing paresthesia associated with beta-alanine consumption when used in combination with the improved beta-alanine compositions described herein. Examples of the anti-inflammatory agent include, but are not limited to hydrocarbon compounds, terpenes, terpenoids, vitamins, hormones, curcumin, fish oil, ginger, resveratrol, spirulina, bromelain, green tea extract, garlic, and amino acids. In certain embodiments, the anti-inflammatory agent is beta-caryophyllene, i.e., a bicyclic sesquiterpene having the structure:

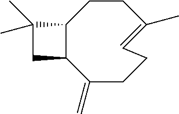

and the chemical name of (1R,4E,9S)-4,11,11-trimethyl-8-methylidenebicycol [7.2.0]undec-4-ene.

Beta-caryophyllene can be obtained from plant-derived oleoresins, essential oils, solutes, distillates, extracts, fermentations, infusions, and leachate from several plants. In certain embodiments, beta-caryophyllene is obtained from essential oils. Examples of the essential oils include, but are not limited to black pepper (Piper nigrum) oil, clove (Syzygium aromaticum) oil, cannabis (Cannabis sativa flower) oil, rosemary oil, hops oil, Cinnamomum tamala oil, Cinnamomum zeylanicum oil, Ocimum micranthum oil, Carum nigrum oil, Lavandula angustifolia oil, Cananga Odorata oil, Humulus lupulus oil, Ocimum gratissimum oil, Origanum vulgare oil, Rosmarinus officinalis oil, Sinularia nanolobata (soft coral) oil, Hyptis fruticose (leaf) oil, Eremanthus erythropappus oil, Senecio rufinervis oil, Baccharis uncinella oil, Copaifera spp. oil, Pterodon emarginatus oil, Aegle marmelos oil, Vitex agnus-castus oil, Ocimum basillicum oil, Valeriana officinalis oil, Thymus vulgaris oil, Salvia Officinalis oil, Zingiber Officinale oil, and the like. In certain embodiments, beta-caryophyllene can be obtained from the stems and/or flowers of clove by steam distillation. The stems and flowers can be dry or fresh. In certain embodiments, beta-caryophyllene is obtained by the following process: dispensing fresh clove (stems and/or flowers) in water, loading the resultant dispensation in a steam distillation unit, steam-distilling the dispensation to give a mixture of oil and water, collecting the mixture, separating the oil from water to give clove oil, transferring the clove oil to a fractional distillation column, and distilling the clove oil to give beta-caryophyllene having at least 98% purity. In certain embodiments, essential oils containing beta-caryophyllene can be used directly without the processing set forth herein. In certain embodiments, clove oil containing between about 15 wt. % and about 25 wt. % of beta-caryophyllene is used. In certain embodiments, clove oil containing about 20 wt. % of beta-caryophyllene is used. In certain embodiments, clove oil containing about 98 wt. % of beta-caryophyllene is used.

An amphiphilic agent, as described herein, may refer to a compound having a hydrophilic group at one end and a lipophilic group at the other end and thus possessing both hydrophilic and lipophilic properties. In certain embodiments, an amphiphilic agent can form a liposome that encapsulates beta-alanine, as described herein. Without being bound by a particular theory, it is believed that the hydrophilic group of the amphiphilic agent can interact with beta-alanine by dipole-dipole and/or ion-dipole forces to form an inner core and the lipophilic group of the amphiphilic agent to form an outer shell to surround the inner core. Without being bound by a particular theory, it is believed that the lipophilic shell can pass through small intestine walls due to its lipophilic nature, which when encapsulating beta-alanine, can aid in the passage of beta-alanine through the small intestine walls where beta-alanine can then be absorbed. Without being bound by a particular theory, it is believed that the lipophilic shell can then pass through cell membranes due to its lipophilic nature as well and carry beta-alanine into the cells, which is a route for beta-alanine to pass through cell membranes in addition to a route via two transporters, i.e., TauT and PAT1. With this additional route, permeability of beta-alanine may be increased and as a result, the amount of beta-alanine in cells increases, which in turn leads to increased bioavailability of beta-alanine and increases in carnosine concentration in the cells. Without being bound by a particular theory, it is believed that such different routes of absorption due to beta-alanine being encapsulated in a liposome can be helpful in reducing paresthesia associated with beta-alanine consumption. Examples of the amphiphilic agents include, but are not limited to phospholipids, cholesterol, glycolipids, fatty acids, bile acids, saponins, and the like. In certain embodiments, an amphiphilic agent, as described herein, is a phospholipid. Phospholipids from both natural and synthetic sources are compatible with compositions described herein and may be used in varying concentrations. Examples of the phospholipids include, are but not limited to dipalmitoylphosphatidylcholine, distearoylphosphatidylcholine, diarachidoylphosphatidylcholine, dibehenoylphosphatidylcholine, diphosphatidyl glycerol, phosphatidylcholines, phosphatidylethanolamines, phosphatidylserines, phosphatidylglycerols, sphingomyelin, lecithin, phosphatidylinositols, and the like. In certain embodiments, the phospholipid, as described herein, is lecithin.

Beta-cyclodextrin is a cyclic oligosaccharide consisting of seven glucose subunits joined by alpha-(2,4)glycosidic bonds forming a truncated conical structure. It is primarily used to enhance the water solubility and bioavailability of poorly water-soluble drugs. It is not used to enhance the water solubility and bioavailability of water-soluble drugs because the unintended interactions between water-soluble drugs and beta-cyclodextrin can lead to complex formation even after dissolution and since beta-cyclodextrin cannot pass the gastrointestinal barrier, the interaction might lead to a delay in absorption of the drugs in the gastrointestinal tract. In certain embodiments, one or more beta-cyclodextrin's derivatives such as 2-hydroxypropyl-β-cyclodextrin, randomly methylated β-cyclodextrin, and β-cyclodextrin sulfobutyl ether sodium salt can be used to replace beta-cyclodextrin or in combination with beta-cyclodextrin.

In certain embodiments, a composition, as described herein, can comprise one or more of an effective amount of beta-alanine, an effective amount of an anti-inflammatory agent, an effective amount of an amphiphilic agent, and an effective amount of beta-cyclodextrin. In certain embodiments, a composition, as described herein, can comprise at least about 1 wt. % of beta-alanine, as described herein, at most about 99 wt. % of an anti-inflammatory agent, as described herein, at most about 99 wt. % of an amphiphilic agent, as described herein, and at most about 90 wt. % of beta-cyclodextrin. In certain embodiments, a composition, as described herein, can comprise between about 10 wt. % and about 99 wt. % of beta-alanine, as described herein, between about 0.01 wt. % and about 50 wt. % of an anti-inflammatory agent, as described herein, between about 0.01 wt. % and about 50 wt. % of an amphiphilic agent, as described herein, and between about 0.01 wt. % and about 50 wt. % of beta-cyclodextrin, as described herein. In certain embodiments, a composition, as described herein, may comprise between about 50 wt. % and about 80 wt. % of beta-alanine, as described herein, between about 0.01 wt. % and about 10 wt. % of an anti-inflammatory agent, as described herein, between about 0.1 wt. % and about 10 wt. % of an amphiphilic agent, as described herein, between about 0.1 wt. % and about 20 wt. % of beta-cyclodextrin, as described herein. In certain embodiments, a composition, as described herein, can comprise between about 70 wt. % and about 80 wt. % of beta-alanine, as described herein, between about 0.1 wt. % and about 5 wt. % of an anti-inflammatory agent, as described herein, between about 1 wt. % and about 5 wt. % of an amphiphilic agent, as described herein, and between about 1 wt. % and about 10 wt. % of beta-cyclodextrin, as described herein. In certain embodiments, a composition, as described herein, can comprise between about 70 wt. % and about 75 wt. % of beta-alanine, as described herein, between about 0.5 wt. % and about 3 wt. % of an anti-inflammatory agent, as described herein, between about 1 wt. % and about 3 wt. % of an amphiphilic agent, as described herein, and between about 4 wt. % and about 8 wt. % of beta-cyclodextrin, as described herein.

In certain embodiments, the amount of beta-alanine and the amount of an anti-inflammatory agent in a composition, as described herein, may be present in a weight ratio ranging from about 150:1 to about 1:150. In certain embodiments, the amount of beta-alanine and the amount of an anti-inflammatory agent in a composition, as described herein, can be present in a weight ratio ranging from about 125:1 to about 1:50. In certain embodiments, the amount of beta-alanine and the amount of an anti-inflammatory agent in a composition, as described herein, may be present in a weight ratio ranging from about 125:1 to about 50:1. In certain embodiments, the amount of beta-alanine and the amount of an anti-inflammatory agent in a composition, as described herein, can be present in a weight ratio ranging from about 125:1 to about 25:1.

In certain embodiments, clove oil containing an anti-inflammatory agent is used in a composition described herein. In certain embodiments, a composition, as described herein, can comprise at least about 1 wt. % of beta-alanine, as described herein, at most about 99 wt. % of clove oil, as described herein, at most about 99 wt. % of an amphiphilic agent, as described herein, and at most 90 wt. % of beta-cyclodextrin. In certain embodiments, a composition, as described herein, can comprise between about 10 wt. % and about 99 wt. % of beta-alanine, as described herein, between about 0.01 wt. % and about 50 wt. % of clove oil, as described herein, between about 0.01 wt. % and about 50 wt. % of an amphiphilic agent, as described herein, and between about 0.01 wt. % and about 50 wt. % of beta-cyclodextrin, as described herein. In certain embodiments, a composition, as described herein, may comprise between about 50 wt. % and about 80 wt. % of beta-alanine, as described herein, between about 0.01 wt. % and about 10 wt. % of clove oil, as described herein, between about 0.1 wt. % and about 10 wt. % of an amphiphilic agent, as described herein, between about 0.1 wt. % and about 20 wt. % of beta-cyclodextrin, as described herein. In certain embodiments, a composition, as described herein, can comprise between about 70 wt. % and about 80 wt. % of beta-alanine, as described herein, between about 0.1 wt. % and about 5 wt. % of clove oil, as described herein, between about 1 wt. % and about 5 wt. % of an amphiphilic agent, as described herein, and between about 1 wt. % and about 10 wt. % of beta-cyclodextrin, as described herein. In certain embodiments, a composition, as described herein, can comprise between about 70 wt. % and about 75 wt. % of beta-alanine, as described herein, between about 1 wt. % and about 5 wt. % of clove oil, as described herein, between about 1 wt. % and about 3 wt. % of an amphiphilic agent, as described herein, and between about 4 wt. % and about 8 wt. % of beta-cyclodextrin, as described herein.

In certain embodiments, the amount of beta-alanine and the amount of clove oil in a composition, as described herein, can be present in a weight ratio ranging from about 50:1 to about 1:1. In certain embodiments, the amount of beta-alanine and the amount of clove oil in a composition, as described herein, can be present in a weight ratio ranging from about 30:1 to about 1:1. In certain embodiments, the amount of beta-alanine and the amount of clove oil in a composition, as described herein, can be present in a weight ratio of about 25:1.

In certain embodiments, the amount of beta-alanine and the amount of an amphiphilic agent in a composition, as described herein, may be present in a weight ratio ranging from about 50:1 to about 1:1. In certain embodiments, the amount of beta-alanine and the amount of an amphiphilic agent in a composition, as described herein, can be present in a weight ratio ranging from about 30:1 to about 10:1. In certain embodiments, the amount of beta-alanine and the amount of an amphiphilic agent in a composition, as described herein, can be present in a weight ratio of about 30:1.

In certain embodiments, the total amounts of beta-alanine, clove oil, and an amphiphilic agent and the amount of beta-cyclodextrin in a composition, as described herein, can be present in a weight ratio ranging from about 50:1 to about 1:1, from about 40:1 to about 1:1, from about 30:1 to 1:1, from about 20:1 to 1:1, from about 15:1 to 8:1, about 12:1, about 11:1, about 10:1, about 9:1, or any values in between.

In certain embodiments, a composition, as described herein, can be coated with a coating agent. Any suitable coating technique may be used, including spray coating, sugar coating, enteric coating, film coating, and the like. In certain embodiments, a coated composition, as described herein, may comprise between about 0.1 wt. % and about 50 wt. %, or between about 0.1 wt. % and about 20 wt. %, or between about 1 wt. % and about 10 wt. %, or between about 2 wt. % and about 5 wt. %, or between about 3 wt. % and about 4 wt. % of a coating agent, and ranges therebetween.

In some embodiments, a coating agent, as described herein, may be a particulate. Suitable coating agents falling within the scope of disclosure include, but are not limited to lactose, cellulose, microcrystalline cellulose, modified food starches, gum Arabic, maltodextrin, dextrose, xanthan gum, guar gum, carboxymethylcellulose, cellulose gel, cellulose gum, sodium caseinate, carrageenan, glucomannan, psyllium, gum acacia, polyethylene glycol, polyvinylpyrrolidone, hydroxypropyl methylcellulose, sodium carboxymethylcellulose, and combinations thereof. In some embodiments, a modified food starch, as described herein, is a starch that is chemically modified by esterification with a C₃-C₂₂ fatty acid chain. In some embodiments, a modified food starch, as described herein, can be a modified corn, potato, wheat, rice, tapioca, sago, oat, barley, amaranth, waxy maize, cassava, waxy barley, waxy rice, glutinous rice, or sweet rice starch. Exemplary modified food starches include, but are not limited to octenyl succinate starches, such as sodium octenyl succinate starch, calcium octenyl succinate starch, and aluminum octenyl succinate starch. A modified food starch, as described herein, can be a modified waxy maize starch, such as a sodium octenyl succinate waxy maize starch. In some embodiments, a modified food starch, as described herein, may be Capsul® (Starch sodium octenyl succinate).

In certain embodiments, a composition, as described herein, may further comprise a stimulant. A stimulant, as described herein, can comprise caffeine, paraxanthine, methylhexanamine, salt thereof, or any combinations thereof. Caffeine can be from a natural source or synthesized. Examples of the natural source can include a green tea extract, a guarana extract, a coffee extract, or a yerba mate extract, lemon argentina, copaiba, orange sweet oil, grapefruit pink, or any combinations thereof. A stimulant, as described herein, can also comprise one or more of acetyl-L-carnitine/L-carnitine, theacrine, arginine, biotin, betel nut, B-vitamins, cacao, cayenne, chia seeds, chlorella, chlorophyll, chromium picolinate, coconut oil, coenzyme Q-10, cordyceps, damiana, dehydroepiandrosterone (DHEA), eluthero, ephedra, Fo-Ti (Polygonum multiflorum), one or more amino acids, Ginkgo biloba, red ginseng, Asian ginseng, Gotu kola/kula, grape seed, holy basil extract, hoodia, Indian ginseng, iodine, L-Theanine, L-Tyrosine, maca, Mucuna pruriens, niacin, Rhodiola rosea, sage, selenium, St. John's Wort, taurine, tea (Cammillia sinensis), Theobroma cacao, tobacco, xanthine, yohimbe, any extract of the foregoing, or a ground powder of any of the foregoing, and the like. An extract can refer to an active ingredient obtained from plants by physical (e.g., pressing) or chemical process (e.g., solvent extraction, distillation, and sublimation). Extracts can be substantially free of solvent, i.e., resulting in an active ingredient in a concentrated form. A ground powder refers to fine, dry particles produced by the grinding, crushing, or disintegration of a solid substance, and the like. In certain embodiments, a composition, as described herein, may comprise between about 0.01 wt. % and about 50 wt. %, or between about 0.05 wt. % and about 20 wt. %, or between about 0.1 wt. % and about 10 wt. %, or between about 0.1 wt. % and about 1 wt. %, or between about 0.1 wt. % and about 0.2 wt. % of a stimulant, and ranges therebetween.

In certain embodiments, the amount of beta-alanine and the amount of an anti-inflammatory agent in a composition are present in a weight ratio ranging from about 10:1 to about 150:1 and the amount of an anti-inflammatory agent and the amount of a stimulant are present in a weight ratio ranging from about 20:1 to about 1:10. In certain embodiments, the amount of beta-alanine and the amount of an anti-inflammatory agent in a composition are present in a weight ratio ranging from about 20:1 to about 125:1 and the amount of an anti-inflammatory agent and the amount of a stimulant are present in a weight ratio ranging from about 20:1 to about 4:1. In certain embodiments, the amount of beta-alanine and the amount of clove oil in a composition are present in a weight ratio ranging from about 20:1 to about 110:1 and the amount of clove oil and the amount of a stimulant are present in a weight ratio ranging from about 20:1 to about 4:1. In certain embodiments, the amount of beta-alanine and the amount of clove oil in a composition are present in a weight ratio ranging from about 20:1 to about 30:1 and the amount of clove oil and the amount of a stimulant are present in a weight ratio ranging from about 20:1 to about 10:1. In certain embodiments, the amount of beta-alanine and the amount of clove oil in a composition are present in a weight ratio of about 25:1 and the amount of clove oil and the amount of a stimulant are present in a weight of about 17:1.

In certain embodiments, the amount of beta-alanine and the amount of an amphiphilic agent in a composition described herein can be present in a weight ratio ranging from about 50:1 to about 1:50 and the amount of an amphiphilic agent and the amount of a stimulant are present in a weight ratio ranging from about 25:1 to about 1:25. In certain embodiments, the amount of beta-alanine and the amount of an amphiphilic agent in a composition as described herein are present in a weight ratio ranging from about 30:1 to about 10:1 and the amount of an amphiphilic agent and the amount of a stimulant are present in a weight ratio ranging from about 25:1 to about 10:1. In certain embodiments, the amount of beta-alanine and the amount of an amphiphilic agent in a composition as described herein are present in a weight ratio of about 30:1 and the amount of an amphiphilic agent and the amount of a stimulant are present in a weight ratio of about 14:1.

In certain embodiments, the amount of beta-alanine and the amount of beta-cyclodextrin in a composition described herein can be present in a weight ratio ranging from about 50:1 to about 1:50 and the amount of beta-cyclodextrin and the amount of a stimulant are present in a weight ratio ranging from about 100:1 to about 1:1. In certain embodiments, the amount of beta-alanine and the amount of beta-cyclodextrin in a composition as described herein are present in a weight ratio ranging from about 50:1 to about 10:1 and the amount of beta-cyclodextrin and the amount of a stimulant are present in a weight ratio ranging from about 50:1 to about 30:1. In certain embodiments, the amount of beta-alanine and the amount of beta-cyclodextrin in a composition as described herein are present in a weight ratio ranging from about 15:1 to about 10:1 and the amount of beta-cyclodextrin and the amount of a stimulant are present in a weight ratio ranging from about 45:1 to about 40:1.

In certain embodiments, a composition, as described herein, may further comprise one or more of HMB. As used herein, HMB can refer to HMB in the free acid form having the chemical name of beta-hydroxy beta-methylbutyric acid (HMB-FA), an ester of HMB-FA, and a salt of HMB-FA, or any combinations thereof. Examples of an ester of HMB-FA include, but are not limited to, methyl ester, ethyl ester, and isopropanol ester of HMB-FA. Examples of a salt of HMB-FA include, but are not limited to, monohydrated calcium salt of HMB-FA (CaHMB).

In certain embodiments, a composition, as described herein, can comprise one or more of an effective amount of beta-alanine, an effective amount of an anti-inflammatory agent, an effective amount of an amphiphilic agent, an effective amount of beta-cyclodextrin, an effective amount of a stimulant, and an effective amount of HMB. In certain embodiments, the amount of beta-alanine and the amount of HMB in a composition described herein can be present in a weight ratio ranging from about 50:1 to about 1:50. In certain embodiments, the amount of beta-alanine and the amount of HMB in a composition as described herein are present in a weight ratio ranging from about 20:1 to about 1:20. In certain embodiments, the amount of beta-alanine and the amount of HMB in a composition as described herein are present in a weight ratio ranging from about 1:1 to about 1:10. In certain embodiments, the amount of beta-alanine and the amount of HMB in a composition as described herein are present in a weight ratio ranging from about 1:1 to about 1:5. In certain embodiments, a composition, as described herein, can comprise at least about 1 wt. % of beta-alanine, as described herein, at most about 99 wt. % of an anti-inflammatory agent, as described herein, at most about 99 wt. % of an amphiphilic agent, as described herein, at most about 90 wt. % of beta-cyclodextrin, at most about 50 wt. % of a stimulant, as described herein, and at most about 99 wt. % of HMB, as described herein. In certain embodiments, a composition, as described herein, can comprise between about 10 wt. % and about 99 wt. % of beta-alanine, as described herein, between about 0.01 wt. % and about 50 wt. % of an anti-inflammatory agent, as described herein, between about 0.01 wt. % and about 50 wt. % of an amphiphilic agent, as described herein, between about 0.01 wt. % and about 1 wt. % of a stimulant, as described herein, between about 0.01 wt. % and about 50 wt. % of beta-cyclodextrin, as described herein, and between about 1.0 wt. % and about 90 wt. % of HMB, as described herein. In certain embodiments, a composition, as described herein, may comprise between about 10 wt. % and about 40 wt. % of beta-alanine, as described herein, between about 0.01 wt. % and about 5 wt. % of an anti-inflammatory agent, as described herein, between about 0.01 wt. % and about 5 wt. % of an amphiphilic agent, as described herein, between about 0.01 wt. % and about 0.1 wt. % of a stimulant, as described herein, between about 0.1 wt. % and about 10 wt. % of beta-cyclodextrin, as described herein, and between about 50 wt. % and about 80 wt. % of HMB, as described herein. In certain embodiments, a composition, as described herein, can comprise between about 15 wt. % and about 30 wt. % of beta-alanine, as described herein, between about 0.1 wt. % and about 1 wt. % of an anti-inflammatory agent, as described herein, between about 0.1 wt. % and about 1 wt. % of an amphiphilic agent, as described herein, between about 0.01 wt. % and about 0.1 wt. % of a stimulant, as described herein, between about 0.5 wt. % and about 5 wt. % of beta-cyclodextrin, as described herein, and between about 60 wt. % and about 75 wt. % of HMB, as described herein. In certain embodiments, a composition, as described herein, can comprise between about 20 wt. % and about 25 wt. % of beta-alanine, as described herein, between about 0.5 wt. % and about 1 wt. % of an anti-inflammatory agent, as described herein, between about 0.5 wt. % and about 1 wt. % of an amphiphilic agent, as described herein, between about 0.01 wt. % and about 0.1 wt. % of a stimulant, as described herein, between about 0.5 wt. % and about 2.0 wt. % of beta-cyclodextrin, as described herein, between 65 wt. % and about 75 wt. % of HMB, as described herein.

The effective amount of beta-alanine in a composition, as described herein, may range from about 0.01 g to about 6400 g, about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, about 0.3 g to 0.6 g, about 0.2 g to 0.5 g, about 0.2 g to 0.4 g, about 0.4 g to 0.5 g, about 0.4 g to 0.6 g, or about 0.2 g to 0.3 g. For example, the amount of beta-alanine in a composition, as described herein, may be about 0.01 g, 0.02 g, 0.03 g, 0.04 g, 0.05 g, 0.06 g, 0.07 g, 0.08 g, 0.09 g, 0.1 g, 0.15 g, 0.16 g, 0.17 g, 0.18 g, 0.19 g, 0.2 g, 0.21 g, 0.22 g, 0.23 g, 0.24 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g, 500 g, 1000 g, 1500 g, 1600 g, 2000 g, 2500 g, 3000 g, 3200 g, 4000 g, 5000 g, 6000 g, 6400 g or more or any amount therebetween.

The amount of beta-alanine can be provided in an effective amount. As set forth herein, and as would be immediately envisaged by the skilled artisan in view of the disclosure contained herein, an effective amount can be an amount capable of achieving the desired outcome while also being provided at a safe amount.

The amount of an anti-inflammatory agent in a composition, as described herein, may be about 0 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575μ g, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The amount of an amphiphilic agent in a composition, as disclosed herein, may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The amount of beta-cyclodextrin in a composition, as disclosed herein, may be about 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175μ, 200μ, 225μ, 250μ, 275μ, 300μ, 325μ, 350μ, 375μ, 400μ, 425μ, 450μ, 475μ g, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The amount of a stimulant in a composition as described herein may be about 0 μg, 1 μg, 2μ, 3μ, 4μ, 5μ, 6μ, 7μ, 8μ, 9μ, 10μ, 15μ, 20μ, 25μ, 30μ, 35μ, 40μ, 45μ g, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The amount of HMB in a composition as described herein may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200μ, 225μ, 250μ, 275μ, 300μ, 325μ, 350μ, 375μ, 400μ, 425μ, 450μ, 475μ g, 500μ, 525μ, 575μ, 600μ, 625μ, 650μ, 675μ, 700μ, 725μ, 750μ, 775μ, 800μ g, 825 μg, 850 μg, 875 μg, 900 μg, 925 μg, 950 μg, 975 μg, 1000 μg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

Compositions described herein may contain any number of additional components, including a diluent, an acid, a base, a lubricant, a binder, disintegrating agents, coloring agents, wetting agents, preservatives, stabilizers, buffer substances, antioxidants, emulsifying agents, suspending agents, solubilizing agents, pH buffering agents, anticaking agents, sweetening agents, flavor agents and the like. Solvents including water, glycerin, sorbitol, ethyl alcohol, syrup and the like can be used in the compositions described herein. Examples of acids include, but are not limited to, tartaric acid, salicylic acid, acetic acid, malic acid, folic acid, fumaric acid, lactic acid, and citric acid. Examples of bases include, but are not limited to, calcium carbonate, trisodium phosphate, potassium bicarbonate, ammonium bicarbonate, calcium hydroxide, sodium benzoate, and sodium bicarbonate. Examples of diluents include, but are not limited to, lactose, sucrose, dicalcium phosphate, and carboxy methylcellulose. Examples of lubricants include, but are not limited, to magnesium stearate, calcium stearate and talc. Examples of binders include, but are not limited to, starch, natural gums (e.g., gum acacia, arabic gum, xanthan gum), gelatin, glucose, molasses, polyvinylpyrrolidone, celluloses and derivatives thereof, povidone, and crospovidone. Examples of coloring agents include, but are not limited to, any of the approved certified water-soluble FD&C dyes. Examples of preservatives include, but are not limited to, glycerin, methyl and propylparaben, benzoic add, potassium sorbate, sodium benzoate and alcohol. Examples of non-aqueous liquids utilized in emulsions include, but are not limited to, mineral oil and cottonseed oil. Examples of emulsifying agents include, but are not limited to, gelatin, acacia, tragacanth, bentonite, and surfactants such as polyoxyethylene sorbitan monooleate. Examples of suspending agents include, but are not limited to, sodium carboxymethylcellulose, pectin, tragacanth, Veegum and acacia. Examples of wetting agents include, but are not limited to, propylene glycol monostearate, sorbitan monooleate, diethylene glycol monolaurate and polyoxyethylene lauryl ether. Examples of pH buffering agents include, but are not limited to, acetate, sodium citrate, cyclodextrine derivatives, sorbitan monolaurate, triethanolamine sodium acetate, and triethanolamine oleate. Examples of anticaking agents include, but are not limited to calcium silicate, stearates of calcium and magnesium, silica and various silicates, talc, flour, starch, and ferrocyanides. Examples of sweetening agents include, but are not limited to, glucose, sucrose, sucralose, syrups, lactitol, maltitol, stevia, xylitol, alcohol sugars, sorbitol, glycerin and artificial sweetening agents such as saccharin, aspartame, and acesulfame potassium. Flavor agents can include natural substances (e.g., wood particles, nut particles, fruit rinds, honey, etc), essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or similar plant material, meat, seafood, poultry, eggs, dairy products, or fermentation products thereof, and the like, whose significant function in food is flavoring rather than nutritional. Flavor agents can also include artificial substances and/or flavorings. Examples of flavoring agents are natural or synthetic vanilla flavor, natural or synthetic chocolate flavor, natural or synthetic coffee flavor, natural or synthetic cocoa flavor, apple fizz flavor, natural or synthetic orange flavor, natural or synthetic lemon lime flavor, citric acid, licorice extract, cinnamon oil, eucalyptus oil, peppermint oil, clove oil, bay oil, thyme oil, lemon oil, lime oil, orange oil, grape oil, grapefruit oil, apple essence, pear essence, peach essence, strawberry essence, watermelon essence, raspberry essence, cherry essence, plum essence, pineapple essence, apricot essence, glycyrrhiza extract, cardamom extract, and the like.

The compositions described herein exhibit unexpectedly high stability (which provides for long shelf-life of dietary supplements described herein) as well as aqueous solubility, aqueous stability, semi-solid solubility and semi-solid stability (which provide their uses for ready-to-drink beverages and as ready-to-make products such as ready-to-make water-based dietary supplements described herein). The compositions described herein exhibit unexpectedly reduced parasthesia sensation experienced by a user when consuming a composition described herein when compared to beta-alanine products in the prior art, and more specifically, free beta-alanine compositions. Compositions described herein can be incorporated into various types of food and beverage products, including, without limitation, still and carbonated soft drinks, energy drinks, sports hydration beverages, ready-to-drink teas and coffees, juice drinks and nectars, flavored waters, dairy-based beverages (e.g., milk drinks, yogurt drinks, shakes), food replacement beverages (e.g., protein shakes, protein drinks, and protein shots), plant-based milks and smoothies, fermented beverages, alcoholic and low-alcohol drinks, sauces and dressings, spreads, confectionery products, baked goods, snack foods, cereals and granola, nutrition bars, dairy and non-dairy desserts (e.g., ice creams, frozen novelties, and puddings), soups and broths, and other solid, semi-solid, or liquid ingestible products suitable for human consumption. As disclosed herein, compositions described herein can be formulated as ready-to-make products (e.g., a powder, a capsule, a tablet that can be mixed into food and beverage compositions or taken along with such compositions food or beverage compositions). In certain embodiments, compositions described herein can be mixed with a protein drink (e.g., a protein shot) before consumption. As used herein, the term “protein drink” can refer to a composition comprising one or more proteins such as marine collagen, bovine collagen, whey protein, whey isolate, and the like as would be understood by the ordinarily skilled artisan.

In certain embodiments, a composition can be in a sustained-release formulation. Sustained release refers to any one of extended-release dosage forms. The following terms may be considered to be substantially equivalent to sustained release, for the purposes of the present disclosure: continuous release, controlled release, delayed release, depot, gradual release, long term release, programmed release, prolonged release, programmed release, proportionate release, protracted release, repository, retarded release, slow release, spaced release, time coat, time release, delayed action, extended action, layered time action, long acting, prolonged action, sustained action medications, extended release, and release in terms of pH level in the gut and intestine.

In certain embodiments, the sustained-release formulation comprises a physical system. Examples of the physical system include, but are not limited to, reservoir systems with rate-controlling membranes such as encapsulation (including micro-encapsulation and macro-encapsulation) and membrane systems; reservoir systems without rate-controlling membranes such as hollow fibers, ultra macroporous cellulose triacetate, and porous polymeric substrates and foams; monolithic systems including systems physically dissolved in non-porous, polymeric, or elastomeric matrices (e.g., non-erodible, erodible, environmental agent ingression, and degradable) and materials physically dispersed in non-porous, polymeric, or elastomeric matrices (e.g., non-erodible, erodible, environmental agent ingression, and degradable); laminated structures including reservoir layers chemically similar or dissimilar to outer control layers; other physical methods such as osmotic pumps or adsorption onto ion-exchange resins and the like.

In certain embodiments, the sustained-release formulation comprises a chemical system. Examples of the chemical system include, but are not limited to, chemical erosion of polymer matrices (e.g., heterogeneous or homogeneous erosion), biological erosion of a polymer matrix (e.g., heterogeneous or homogeneous), hydrogels and the like.

In certain embodiments, the sustained-release formulation is a microencapsulated powder; a osmotic pressure-controlled gastrointestinal delivery system; a hydrodynamic pressure-controlled gastrointestinal delivery system; a membrane permeation-controlled gastrointestinal delivery system, including microporous membrane permeation-controlled gastrointestinal delivery devices; a gel diffusion controlled gastrointestinal delivery system; and an ion-exchange-controlled gastrointestinal delivery system, including cationic and anionic drugs and the like. In one embodiment, the sustained-release formulation can be an oil-microencapsulated sustained release powder dosage form that can be mixed with liquid and consumed as a drink mix beverage.

In certain embodiments, the sustained-release formulation can be obtained by using sufficient amount of the conventional pharmaceutical binders, excipients and additives as sustained-release polymers to produce a sustained-release effect. These include, but are not limited to, gelatin, natural sugars (e.g., raw sugar and lactose), lecithin, mucilage, plant gums, pectins, pectin derivatives, algal polysaccharides, glucomannan, agar, lignin, guar gum, locust bean gum, acacia gum, xanthan gum, carrageenan gum, karaya gum, tragacanth gum, ghatti gum, starches (e.g., corn starch and amylose), dextran, polyvinyl pyrrolidone, polyvinyl acetate, gum arabic, alginic acid, tylose, talcum, lycopodium, silica gel (e.g., colloidal), cellulose, cellulose derivatives (e.g., cellulose ethers, methoxypropyl cellulose, methyl cellulose, hydroxypropyl methyl cellulose, hydroxypropyl methyl cellulose phthalate, cross-linked sodium carboxymethylcellulose, cross-linked hydroxypropylcellulose, high-molecular weight hydroxymethylpropylcellulose, carboxymethylcellulose, low-molecular weight hydroxypropylmethylcellulose medium-viscosity hydroxypropylcellulose, hydroxypropylmethylcellulose hydroxyethylcellulose, hydroxypropylmethylcellulose, sodium carboxymethylcellulose, alkyl celluloses, ethyl cellulose, cellulose acetate, cellulose propionate, cellulose acetatepropionate, cellulose acetate butyrate, cellulose triacetate, methyl cellulose, hydroxypropyl cellulose, and hydroxypropylmethyl cellulose), fatty acids including saturated fatty acids (e.g., stearates such as magnesium stearate), glycerol esters and polyglycerol esters of saturated fatty acids and their mixtures, magnesium salts of fatty acids, calcium salts of fatty acids, aluminum salts of fatty acids, polycarboxylic acids, emulsifiers, oils and fats including vegetable oils (e.g., peanut oil, castor oil, olive oil, sesame oil, cottonseed oil, corn oil, wheat germ oil, sunflower seed oil, and cod fiver oil), hydrogenated vegetable oils suitable for microencapsulation, pharmaceutically acceptable monovalent or multivalent alcohols and polyglycols such as polyethylene glycol and derivatives thereof, esters of aliphatic saturated or unsaturated fatty acids with monovalent aliphatic alcohols, multivalent alcohols (e.g., glycols, glycerol, diethylene glycol, pentacrythritol, sorbitol, and mannitol), esterified monovalent aliphatic alcohols, esterified multivalent alcohols, esters of citric acid with primary alcohols, acetic acid, urea, benzyl benzoate, dioxolanes, glycerofonnals, tetrahydrofurfuryl alcohol, polyglycol ethers, dimethylacetamide, lactamides, lactates, ethylcarbonates, silicones (e.g., medium-viscous polydimethyl siloxanes), calcium carbonate, sodium carbonate, calcium phosphate, sodium phosphate, magnesium carbonate and the like.

In certain embodiments, the sustained-release formulation can be obtained by encapsulating beta-alanine with one or more coating agents to create or enhance the sustained release features of the composition. For example, plasticizers that are useful as coating agents include, but are not limited to, citric and tartaric acid esters (e.g., acetyl-triethyl citrate, acetyl tributyl-citrate, tributyl-citrate, and triethyl-citrate); glycerol and glycerol esters (e.g., glyceroldiacetate, glyceroltriacetate, acetylated monoglycerides, and castor oil); phthalic acid esters (e.g., dibutyl-phthalate, diamyl-phthalate, diethyl-phthalate, dimethyl-phthalate, dipropyl-phthalate), di-(2-methoxy- or 2-ethoxyethyl)-phthalate, ethylphthalyl glycolate, butylphthalylethyl glycolate, and butylglycolate; alcohols (e.g., propylene glycol, and polyethylene glycol of various chain lengths); adipates (e.g., diethyladipate, di (2-methoxy)-adipate, and di(2-ethoxyethyl)-adipate); benzophenone; diethyl-sebacate; diburylsebacate; dibutylsuccinate; dibutyltartrate; diethylene glycol dipropionate; ethyleneglycol diacetate; ethyleneglycol dibutyrate; ethyleneglycol dipropionate; tributyl phosphate; tributyrin; polyethylene glycol sorbitan monooleate; polysorbates such as Polysorbar 50; sorbitan monooleate and the like.

Certain embodiments comprise a beta-alanine composition comprising an encapsulated beta-alanine composition and an unencapsulated beta-alanine composition as described herein. As described herein, an encapsulated beta-alanine composition can refer to beta-alanine that is substantially present as encapsulated within an amphiphilic agent. As described herein, an unencapsulated beta-alanine composition can refer to beta-alanine that is not encapsulated with an amphiphilic agent. It is believed that a beta-alanine composition comprising an encapsulated beta-alanine composition and an unencapsulated beta-alanine composition has a dual effect; for example, an encapsulated beta-alanine composition can increase the amount of beta-alanine in cells/tissues in a short period of time and thus provides an instant effect and an unencapsulated beta-alanine composition can provide an additional effect, for example, a constant amount of beta-alanine in the cells/tissues and thus provides a long-lasting effect, for example, when provided as in an unencapsulated sustained release formulation. As described herein, both an encapsulated beta-alanine composition and an unencapsulated beta-alanine composition can be in sustained-release formulations. In certain embodiments, a beta-alanine composition comprises an encapsulated beta-alanine composition in a conventional-release formulation and an unencapsulated beta-alanine composition in a sustained-release formulation. In certain embodiments, a beta-alanine composition comprises an encapsulated beta-alanine composition in a sustained-release formulation and an unencapsulated beta-alanine composition in a conventional-release formulation. In certain embodiments, a beta-alanine composition comprises an encapsulated beta-alanine composition and an unencapsulated beta-alanine composition both in sustained-release formulations. In certain embodiments, a beta-alanine composition comprises an encapsulated beta-alanine composition and an unencapsulated beta-alanine composition both in conventional-release formulations. Conventional release refers to a beta-alanine composition that is not formulated as sustained release, as described herein and can refer to compositions wherein no deliberate effort is made to modify the beta-alanine release rate.

In some embodiments, a composition described herein can comprise an effective amount of an encapsulated beta-alanine and an effective amount of an unencapsulated beta-alanine. In certain embodiments, a composition comprises at least 1 wt. % an encapsulated beta-alanine and at most 99 wt. % an unencapsulated beta-alanine. In certain embodiments, a composition comprises at most 99 wt. % an encapsulated beta-alanine and at least 1 wt. % an unencapsulated beta-alanine. In certain embodiments, the amount of an encapsulated beta-alanine and the amount of an unencapsulated beta-alanine in a composition can be present in a weight ratio ranging from 99:1 to 1:99. In certain embodiments, the amount of an encapsulated beta-alanine and the amount of an unencapsulated beta-alanine in a composition can be present in a weight ratio ranging from 9:1 to 1:9. In certain embodiments, the amount of an encapsulated beta-alanine and the amount of an unencapsulated beta-alanine in a composition can be present in a weight ratio ranging from 4:1 to 1:4. In certain embodiments, the amount of an encapsulated beta-alanine and the amount of an unencapsulated beta-alanine in a composition can be present in a weight ratio ranging from 7:3 to 3:7. In certain embodiments, the amount of an encapsulated beta-alanine and the amount of an unencapsulated beta-alanine in a composition can be present in a weight ratio ranging from 3:2 to 2:3. In certain embodiments, the amount of an encapsulated beta-alanine and the amount of an unencapsulated beta-alanine in a composition can be present in a weight ratio of 1:1.

The amount of an unencapsulated beta-alanine and the amount of an encapsulated beta-alanine in a composition independently may be about 0.005 g, 0.01 g, 0.02 g, 0.03 g, 0.04 g, 0.05 g, 0.06 g, 0.07 g, 0.08 g, 0.09 g, 0.1 g, 0.15 g, 0.16 g, 0.17 g, 0.18 g, 0.19 g, 0.2 g, 0.21 g, 0.22 g, 0.23 g, 0.24 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g, 500 g, 1000 g, 1500 g, 1600 g, 2000 g, 2500 g, 3000 g, 3200 g, 4000 g, 5000 g, 6000 g, 6400 g or more or any amount therebetween.

In certain embodiments, a composition as described herein may be formulated as a pharmaceutical composition for humans and animals. The pharmaceutical composition may comprise a pharmaceutically acceptable vehicle, carrier, or diluent. Pharmaceutically acceptable vehicle, carriers and diluent are known in the art and their application would be immediately envisaged by the skilled artisan in view of the disclosure contained herein, and their selections usually are dependent upon the intended route of delivery. The pharmaceutical composition can be liquids, semi-liquids, capsules, tablets, pills, powders, granules, sterile parenteral solutions or suspensions, oral solutions or suspensions, oil water emulsions, implants and microencapsulated delivery systems, and biodegradable, biocompatible polymers such as collagen, ethylene vinyl acetate, polyanhydrides, polyglycolic acid, polyorthoesters, and polylactic acid. Carriers for a liquid composition include, for example, water, saline, aqueous dextrose, glycerol, glycols, ethanol, and the like. Modes of delivery of the pharmaceutical compositions include oral delivery, mucosal delivery, nasal delivery, ocular delivery, transdermal delivery, parenteral delivery, vaginal delivery, rectal delivery, intrauterine delivery and the like.

In certain embodiments, a composition as described herein can be formulated as a dietary supplement for humans and animals. Formulations of a dietary supplement can include, but is not limited to, tablets, capsules, granules, sachet, gummy, powders, gel, a water-based beverage such as a sports drink, a soft drink (carbonated and non-carbonated), food and beverage compositions, functional foods, medical foods, a food ingredient, or the like. Types of oral tablets include compressed, chewable lozenges and tablets which may be enteric coated, sugar coated, film coated or the like. Capsules can be hard gelatin capsules, soft gelatin capsules, or vegetarian capsules (such as hard HPMC capsules). By way of example, a composition described herein can be suspended in, for example, propylene carbonate, vegetable oils and/or triglycerides and encapsulated in a gelatin capsule. Granules and powders can be provided in a non-effervescent or an effervescent form with a combination of other ingredients known to those skilled in the art. Sachets can have compositions described herein as solids or as liquids. For example, compositions described herein can be formulated as liquids with pH values of about 4-5 and specific gravity of about 1-2 g/ml which are then provided as sachets.

In certain embodiments, compositions described herein can be administered once a day, twice a day, three times a day, four times a day, five times a day, six times a day, seven times a day, eight times a day, nine times a day, ten times a day, or more times a day.

In certain embodiments, a dietary supplement contains about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to about 0.8 g, about 0.3 g to about 0.6 g, about 0.2 g to about 0.5 g, about 0.2 g to about 0.4 g, about 0.4 g to about 0.5 g, about 0.4 g to about 0.6 g, or about 0.2 g to about 0.3 g, or any ranges therebetween of beta-alanine. In certain embodiments, a human dietary supplement contains about 0.1 g, 0.15 g, 0.2 g, 0.21 g, 0.22 g, 0.23 g, 0.24 g, 0.25 g, 0.26 g, 0.27 g, 0.28 g, 0.29 g, 0.3 g, 0.31 g, 0.32 g, 0.33 g, 0.34 g, 0.35 g, 0.36 g, 0.37 g, 0.38 g, 0.39 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g of beta-alanine. In certain embodiments, a human dietary supplement contains about 0 mg to about 10 g, about 1 mg to about 1 g, about 2 mg to about 500 mg, about 3 mg to about 350 mg, about 4 mg to about 150 mg, about 5 mg to about 100 mg, about 6 mg to about 50 mg, about 7 mg to about 25 mg, about 2 mg to about 4 mg, or about 10 mg to about 20 mg of an anti-inflammatory agent. In certain embodiments, a human dietary supplement contains about 0 mg to about 10 g, about 0.1 mg to about 1 g, about 1 mg to about 500 mg, about 3 mg to about 250 mg, about 5 mg to about 150 mg, about 9 mg to about 100 mg, or about 10 mg to about 20 mg of an amphiphilic agent. In certain embodiments, a human dietary supplement contains about 0 mg to about 10 g, about 10 mg to about 1 g, about 20 mg to about 500 mg, about 30 mg to about 250 mg, about 40 mg to about 150 mg, about 50 mg to about 100 mg, or about 0.1 mg to about 1 mg of a stimulant. In certain embodiments, a human dietary supplement may contain about 0 g to about 10 g, about 0.1 g to about 6.0 g, about 0.2 g to about 4.0 g, about 0.5 g to about 3.0 g, about 1.0 g to about 2.0 g, or more and any values therebetween of HMB. In certain embodiments, a human dietary supplement described herein is administered twice a day.

In certain embodiments, a dietary supplement described herein may include other ingredients such as amino acids, proteins (e.g., collagen proteins such as marine collagen proteins and non-marine animal collagen proteins, whey protein, whey protein isolate, pea protein, hemp protein, brown rice protein, soy protein, egg white protein, and casein protein), carbohydrates (e.g., starch and/or sugars, e.g., glucose, fructose, sucrose, and maltrose), insulin, insulin mimics, insulin-action modifier, glycine, fiber, fat, creatine, beta-alanylhistidine peptides (e.g. carnosine, anserine, and/or balenine), galactose, electrolytes, vitamins (e.g., vitamin E, vitamin C, thiamin, riboflavin, niacin, vitamin B6, folic acid, vitamin B12, biotin, and pantothenic acid), lipids, trace elements (e.g., calcium and potassium in the form of their gluconates, phosphates or hydrogen phosphates, and magnesium as the oxide or carbonate, chromium as chromium picolinate, selenium as sodium selenite or selenate, and zinc as zinc gluconate), colorings, natural health improving substances, probiotics, anti-oxidants (e.g., green tea polyphenols and grape seed extract), stabilizers, preservatives, alpha-lipoic acid, tocotrienols, N-acetylcysteine, co-enzyme Q-10, rosemary extracts, COX-1 type inhibitors (e.g., resveratrol), Ginkgo biloba, garlic extracts, buffers and the like.

A composition disclosed herein can be administered to a subject to prevent, treat, maintain healthy levels of, or alleviate declined brain functions, mood disorders, and mental fatigue. Brain functions include, but are not limited to, executive function, mental clarity, cognitive function, memory, attention, task-focus, concentration, alertness, and responses to everyday stress. Declined brain functions can be caused by aging, which include, but are not limited to, dementia, memory decline, and decline in cognitive abilities. Mood disorders refer to emotional state or a mood that is distorted or inconsistent with a subject's circumstances and interferes with a subject's ability to function. Examples of mood disorders include, but are not limited to, depression, anxiety, major depression, dysthymia (dysthymic disorder), bipolar disorder, mood disorder linked to another health condition, mood disorders due to aging (e.g., age-related depression and anxiety), and substance-induced mood disorder. In certain embodiments, a composition described herein can be administered to a subject to maintain healthy levels of brain functions and mood balance. In certain embodiments, a composition described herein can be administered to a subject to restore brain functions and mood balance to healthy levels. In certain embodiments, a composition described herein can be administered to a subject to improve executive function and mental clarity, reduce mental fatigue, improve cognitive function, prevent memory loss, improve memory, enhance mental acuity, improve attention and task focus, improve concentration and alertness, promote healthy responses to everyday stress, foster balanced mood, and maintain calmness under pressure. In certain embodiments, a composition described herein can be administered to a subject to prevent, treat, maintain healthy levels of, or alleviate declined brain functions (e.g., dementia, memory decline, and a decline in cognitive abilities), mood disorders (e.g., depression and anxiety), and mental fatigue caused by aging. In certain embodiments, a composition described herein can be administered to a subject to maintain healthy levels of brain functions and mood balance during aging. In certain embodiments, a composition described herein can be administered to a subject to restore brain functions and mood balance to healthy levels, improve executive function and mental clarity, reduce mental fatigue, improve cognitive function, prevent dementia, prevent memory loss, improve memory, improve attention and task focus, improve concentration and alertness, promote healthy responses to everyday stress, foster balanced mood, reduce anxiety, and maintain calmness under pressure.

A composition disclosed herein can be administered to a subject to prevent, treat or alleviate a weakened immune system. A composition described herein can also be administered to a subject to maintain a healthy immune system and/or restore weaken immune system to heathy levels. A weakened immune system can be caused by aging, among other factors. In certain embodiments, a composition described herein can be administered to a subject to maintain and support healthy immune function and structures. Healthy immune function refers to healthy immune response and everyday wellness. In certain embodiments, a composition described herein can be administered to a subject to prevent and/or alleviate oxidative damages in cells. In certain embodiments, a composition described herein can be administered to a subject to promote healthy aging. In certain embodiments, a composition described herein can be administered to a subject to attenuate the aging process. In certain embodiments, a composition described herein can be administered to a subject to slow down age-related changes, including, but are not limited to, memory decline, drier skin, a decline in cognitive abilities, age-related muscle atrophy, muscle wasting, sarcopenia, age-related bone changes (e.g., bone loss, bone brittleness, bone spurs, shorter trunk and spine, less pronounced foot arches, joint breakdown, and increased risk of fractures), cartilage wear and tear, depression, anxiety, dementia, increased risk of heart diseases, and increased risk of diabetes.

A composition disclosed herein can be administered to a subject to prevent, treat, or alleviate declined muscle strength and/or bone/joint dysfunctions, which may be caused by aging, among other factors. A composition disclosed herein can also be administered to a subject to maintain healthy muscle strength (such as a healthy level of muscle mass) and/or bone/joint functions and/or restore muscle strength and bone/joint functions to healthy levels. As muscle mass closely correlates to physical performance, a composition disclosed herein can be administered to a subject to maintain a healthy level of physical performance. Examples of declined muscle strength include, but are not limited to, muscle fatigue and loss of muscle mass. Examples of bone dysfunctions include, but are not limited to, low bone density, osteoporosis, osteogenesis imperfecta, Paget's disease of bone, cancer, infections, and other bone diseases caused by aging, poor nutrition, genetics, or problems with the rate of bone growth or rebuilding. A subject may have low bone mass at any age but not develop osteoporosis. Examples of joint dysfunctions include, but are not limited to, arthritis (e.g., osteoarthritis) and bursitis. In certain embodiments, a composition can be administered to a subject to increase muscle strength and/or working capacity, improve endurance, and/or delaying the onset of muscle fatigue during exercise or physical exertion, including everyday tasks that become more difficult with age along with high-intensity exercise. Work capacity refers to the ability to perform real physical work and can be assessed by aerobic capacity, endurance, energetic efficiency, voluntary activity, and work productivity. Endurance is defined as the maximum length of time a subject can sustain a given workload without rest. In certain embodiments, a composition can be administered to a subject to prevent bone loss or restore bone density to healthy levels. In certain embodiments, a composition can be administered to a subject to maintain healthy joints or restore joint to healthy levels. In certain embodiments, a composition described herein can be administered to a subject to maintain healthy levels of muscle mass, function, quality, strength, and working capacity during aging. In certain embodiments, a composition described herein can be administered to a subject to stimulate bone growth, maintain healthy levels of bone mass, or reduce bone loss. In certain embodiments, a composition described here can be administered to a subject to maintain healthy levels of bone mass during aging. In certain embodiments, a composition can be administered to a subject to maintain joint health during aging. In certain embodiments, a composition described herein can be administered to a subject to alleviate age-related muscle loss, bone density loss, and cartilage wear and tear. In certain embodiments, a composition can be administered to a subject age 30 or older to promote a healthy level of physical performance.

A composition disclosed herein can be administered to a subject to prevent and treat diabetes and cardiovascular diseases. In certain embodiments, a composition disclosed herein can be administered to an overweight and obese subject to improve the plasma lipidome. In certain embodiments, a composition disclosed herein can be administered to an overweight and obese subject to normalize the serum adipokine concentrations. In certain embodiments, a composition disclosed herein can be administered to a subject to maintain healthy blood sugar levels. In certain embodiments, a composition disclosed herein can be administered to a subject to improve contractility and cardiac function of heart and blood circulation. In certain embodiments, a composition disclosed herein can be administered to a subject to maintain healthy levels of contractility and cardiac function of heart and blood circulation.

Also described herein is a method of increasing athletic performance in a subject without causing paresthesia or with reduced paresthesia effect. The method comprises administering to the subject an effective amount of a composition described herein to safely and effectively increase the synthesis of carnosine in muscle, to attenuate metabolic acidosis and muscle fatigue during physical exercise, and to obviate the paresthesia sensation. Examples of increased athletic performance include improved stamina, speed, strength, power, endurance, flexibility, agility, and balance, focus coordination, reaction time, and fatigue recovery and the like.

A method of improving cognitive function in a subject is also provided herein. The method comprises administering to the subject an effective amount of a composition described herein to safely and effectively increase the concentration of carnosine in brain and to obviate the paresthesia sensation. Improved cognitive function includes improved attention span, memory, reaction time, problem-solving ability, and mental alertness. In certain embodiments, the subject is an adult age 30 or older. Subjects being administered compositions described herein for the purposes described herein can be over the age of 40, over the age of 50, over the age of 60, over the age of 65, over the age of 70, over the age of 80, and over the age of 90.

Methods of attenuating the aging process (i.e., anti-aging functions) and promoting healthy aging and general wellness (including preventing age-related muscle atrophy, and maintaining a healthy level of muscle mass and physical performance) are also provided herein. The methods each comprise administering to a subject an effective amount of a composition described herein to safely and effectively increase carnosine concentrations in tissues in the body (including skeletal muscle, heart muscle, brain, and cells). In certain embodiments, the subject is an adult age 30 or older. Subjects being administered compositions described herein for the purposes described herein can be over the age of 40, over the age of 50, over the age of 60, over the age of 65, over the age of 70, over the age of 80, and over the age of 90.

A composition can be administered in a single dose or multiple doses per day. For example, a composition can be administered one time to twenty-four times in a 24-hour period. In certain embodiments, a composition can be administered twice a day. In certain embodiments, the total dosage of beta-alanine in a composition described herein for a 24-hour period may be about 0.1 g, 0.2 g, 0.21 g, 0.22 g, 0.23 g, 0.24 g, 0.25 g, 0.26 g, 0.27 g, 0.28 g, 0.29 g, 0.3 g, 0.31 g, 0.32 g, 0.33 g, 0.34 g, 0.35 g, 0.36 g, 0.37 g, 0.38 g, 0.39 g, 0.4 g, 0.41 g, 0.42 g, 0.43 g, 0.44 g, 0.45 g, 0.46 g, 0.47 g, 0.48 g, 0.49 g, 0.5 g, 0.51 g, 0.52 g, 0.53 g, 0.54 g, 0.55 g, 0.56 g, 0.57 g, 0.58 g, 0.59 g, 0.6 g, 0.7 g, 0.8 g, 0.9 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g or more or any amount therebetween. In certain embodiments, a composition described herein contains about 0.01 g to about 6.4 g, about 0.04 g to about 3.2 g, about 0.05 g to about 2.24 g, about 0.1 g to about 1 g, about 0.2 g to 0.4 g, about 0.4 g to 0.5 g, about 0.4 g to 0.6 g, about 0.3 g to about 0.6 g, or about 0.2 g to about 0.8 g, or more and any values therebetween of beta-alanine per serving. In certain embodiments, a composition described herein contains about 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g of beta-alanine per serving. In certain embodiments, a composition described herein contains about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to about 0.4 g, about 0.4 g to about 0.5 g, about 0.4 g to about 0.6 g, about 0.3 g to about 0.6 g, or about 0.2 g to about 0.8 g of beta-alanine, or more and values therebetween, administered in a 24-hour period. In certain embodiments, a composition described herein contains about 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, or more and values therebetween of beta-alanine administered in a 24-hour period.

The total dosage of an anti-inflammatory agent in a composition described herein for a 24-hour period may be about 0 μg to about 10 g, about 0.01 g to about 4 g, about 0.01 g to about 2.0 g, about 0.01 g to about 1.0 g, about 0.01 g to about 0.5 g, about 0.01 g to about 0.1 g, about 0.01 g to about 0.05 g, about 0.01 g to about 0.04 g, or about 0.01 g to about 0.03 g, or more and values therebetween. For example, the total dosage of an anti-inflammatory agent in a composition described herein for a 24-hour period may be about 0 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of an amphiphilic agent in a composition described herein for a 24-hour period may be about 0 μg to about 10 g, about 0.01 g to about 4 g, about 0.01 g to about 2.0 g, about 0.01 g to about 1.0 g, about 0.01 g to about 0.5 g, about 0.01 g to about 0.1 g, about 0.01 g to about 0.05 g, about 0.01 g to about 0.04 g, or about 0.01 g to about 0.03 g, or more and values therebetween. For example, the total dosage of an amphiphilic agent in a composition described herein for a 24-hour period may be about 0 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of a stimulant in a composition described herein for a 24-hour period may be about 0 μg to about 10 g, about 0.001 g to about 4 g, about 0.001 g to about 2.0 g, about 0.001 g to about 1.0 g, about 0.001 g to about 0.5 g, about 0.001 g to about 0.1 g, about 0.001 g to about 0.05 g, about 0.001 g to about 0.04 g, about 0.001 g to about 0.03 g, about 0.002 g to 0.01 g, about 0.003 g to about 0.008 g, about 0.004 g to about 0.006 g, or any values therebetween. For example, the total dosage of a stimulant in a composition described herein for a 24-hour period may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.1 mg, 1.2 mg, 1.3 mg, 1.4 mg, 1.5 mg, 1.6 mg, 1.7 mg, 1.8 mg, 1.9 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of HMB in a composition described herein for a 24-hour period may be about 0 g to about 10 g, about 0.1 g to about 6.0 g, about 0.2 g to about 4.0 g, about 0.5 g to about 3.0 g, about 1.0 g to about 2.0 g, or more and any values therebetween. For example, the total dosage of HMB in a composition described herein for a 24-hour period may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225μ, 250μ, 275μ, 300μ, 325μ, 350μ, 375μ, 400μ, 425μ, 450μ, 475μ, 500μ, 525μ g, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.1 mg, 1.2 mg, 1.3 mg, 1.4 mg, 1.5 mg, 1.6 mg, 1.7 mg, 1.8 mg, 1.9 mg, 2 mg, 3 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

A composition described herein for improving athletic performance can be given on a daily basis over a period of about 1 day to about four or more weeks. In certain embodiments, a composition described herein can be administered for about 28 days. In certain embodiments, a composition described herein can be administered for at least 28 consecutive days. In certain embodiments, a composition described herein can be administered to slow down aging and/or support general wellness for subjects over the age of 30, over the age of 40, over the age of 50, over the age of 60, over the age of 70, over the age of 80, or over the age of 90. A composition described herein can be administered in a single dose or multiple doses per day and the total dosage of beta-alanine in the composition for each day of administration may be about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to about 0.8 g, about 0.2 g to about 0.4 g, about 0.4 g to about 0.5 g, about 0.4 g to about 0.6 g, or about 0.3 g to about 0.6 g.

The amount of a composition described herein administered can be adjusted daily, weekly, or monthly. In certain embodiments, the amount of a composition administered can be increased daily, every two days, every three days, every four days, every five days, and every six days, or longer. In certain embodiments, the amount of a composition administered can be increased weekly, every two weeks, every three weeks, or every four weeks. In certain embodiments, the amount of a composition administered can be increased daily for about two days, three days, four days, five days, six days, seven days, fourteen days, twenty-one days, twenty-eight days, thirty days, sixty days, or more or any days in between, followed by a constant dosage, a decreased dosage, or a combination thereof. In certain embodiments, a composition can be administered in treatment periods that last for about four weeks. In certain embodiments, a composition that does not contain an anti-inflammatory agent may be administered first followed by administration of an anti-inflammatory agent-containing composition, where the interval between the two administrations can be from about 15 minutes to 24 hours. In certain embodiments, a composition that does not contain an anti-inflammatory agent and an anti-inflammatory agent-containing composition can be administered alternately.

A beta-alanine composition comprising an encapsulated beta-alanine composition and an unencapsulated beta-alanine composition can be administered. In certain embodiments, a composition comprises an unencapsulated beta-alanine and an encapsulated beta-alanine at a total amount of about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g or about 0.2 g to 0.4 g, and values therebetween, of beta-alanine per serving. In certain embodiments, a composition comprises an unencapsulated beta-alanine and an encapsulated beta-alanine at a total amount of about 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween, of an unencapsulated beta-alanine and an encapsulated beta-alanine per serving. The total dosage of an unencapsulated beta-alanine and an encapsulated beta-alanine in a composition for each day may be about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, or about 0.3 g to 0.6 g and values therebetween.

Certain dosing regimens can comprise the administration of alternating dosages of encapsulated and unencapsulated beta-alanine. In certain embodiments, and as one example of alternating days of administration, the total dosage of an unencapsulated beta-alanine and an encapsulated beta-alanine in a composition for an odd number date of a month may be about 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween, and for an even number date of the month is different from that on the odd date and may be about 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween. Alternating dosages of encapsulated and unencapsulated beta-alanine can comprise alternating dosages within a single day, every other day, or other variations of alternating dosage regimes that would be immediately envisaged by the skilled artisan in view of the disclosure contained herein. The dosing regimens described herein are not particularly limited and can comprise dosing regimens wherein an encapsulated beta-alanine is taken in the same day as an unencapsulated beta-alanine and regimens wherein unencapsulated beta-alanine is not taken in the same day as an encapsulated beta-alanine, and vice versa.

In certain embodiments, an encapsulated beta-alanine composition and an unencapsulated beta-alanine composition can be administered separately. In certain embodiments, an encapsulated beta-alanine may be administered to a subject at the same time as an unencapsulated beta-alanine. In those instances, the encapsulated beta-alanine and unencapsulated beta-alanine may be administered in a single composition or in more than one composition that are administer in temporal proximity to one another. The relative amounts of encapsulated beta-alanine and unencapsulated beta-alanine can be administered to subjects in any of the amounts and ratios as described herein and those that would be immediately envisaged by the skilled artisan in view of the disclosure contained herein. In certain embodiments, an encapsulated beta-alanine composition may be administered first followed by administration of an unencapsulated beta-alanine composition, where the interval between the two administrations can be from about 15 minutes to 24 hours. In certain embodiments, an unencapsulated beta-alanine composition may be administered first followed by administration of an encapsulated beta-alanine composition, where the interval between the two administrations can be from about 5 minutes to about 24 hours, and any interval therebetween. In certain embodiments, an encapsulated beta-alanine composition and an unencapsulated beta-alanine composition can be administered alternately. In certain embodiments, an encapsulated beta-alanine composition may be administered to a subject at a different time or frequency than an unencapsulated beta-alanine. For example, and without limitation, an encapsulated beta-alanine may be administered to a subject several times per week whereas an unencapsulated beta-alanine may be administered daily. As another example, and without limitation, an unencapsulated beta-alanine may be administered to a subject several times per week whereas an encapsulated beta-alanine may be administered daily. In such instances, the unencapsulated beta-alanine may be present in a sustained release, or other controlled or delayed release formulation. Each form of beta-alanine can be administered at the frequencies described herein. In certain embodiments, an unencapsulated beta-alanine composition and an encapsulated beta-alanine composition independently contain about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, or about 0.2 g to 0.4 g and values therebetween, of beta-alanine. In certain embodiments, an unencapsulated beta-alanine composition and an encapsulated beta-alanine composition independently contain about 0.05 g, 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween, of beta-alanine. The total dosage of an unencapsulated beta-alanine and an encapsulated beta-alanine from two compositions for each day may be about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, or about 0.3 g to 0.6 g and values therebetween.

Compositions described herein may be administered alone or in conjunction with other supplements, vitamins, or drugs useful in enhancing athletic performance and/or reducing paresthesia. Compositions described herein can be administered alone or in conjunction with other supplements, vitamins, or drugs to maintain healthy levels of brain activity, cognition, memory, mental acuity, focus, and spatial awareness.

A composition described herein can be administered in a single dose or multiple doses per day. For example, a composition can be administered one time, twice, three times, four times, five times, six times, seven times, eight times or more in a 24-hour period. In certain embodiments, the total dosage of beta-alanine in a composition for a 24-hour period may be about 0.01 g to about 6400 g, about 0.02 g to about 10 g, about 0.03 g to about 6.4 g, about 0.04 g to about 3.2 g, about 0.05 g to about 2.24 g, about 0.1 g to about 1 g, about 0.2 g to about 0.8 g, about 0.2 g to about 0.4 g, about 0.4 g to about 0.5 g, about 0.4 g to about 0.6 g, or about 0.3 g to about 0.6 g, or more and any values therebetween. For example, the total dosage of beta-alanine in a composition for a 24-hour period may be about 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.3 g, 1.4 g, 1.5 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g, 500 g, 1000 g, 1500 g, 1600 g, 2000 g, 2500 g, 3000 g, 3200 g, 4000 g, 5000 g, 6000 g, 6400 g or more or any amount therebetween.

The total dosage of an anti-inflammatory agent in a composition for a 24-hour period may be about 0 μg to about 10 g, about 0.01 g to about 4 g, about 0.01 g to about 2.0 g, about 0.01 g to about 1.0 g, about 0.01 g to about 0.5 g, about 0.01 g to about 0.1 g, about 0.01 g to about 0.05 g, about 0.01 g to about 0.04 g, or about 0.01 g to about 0.03 g, or more and values therebetween. For example, the total dosage of an anti-inflammatory agent in a composition for a 24-hour period may be about 0 μg, 10 μg, 15 μg, 20 μg, 25 μg, 30 μg, 35 μg, 40 μg, 45 μg, 50 μg, 55 μg, 60 μg, 65 μg, 70μ, 75μ, 80μ, 85μ, 90μ, 95μ, 100μ, 125μ, 150μ, 175μ, 200μ, 225μ, 250μ, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.8 g, 2.0 g, 2.25 g, 2.4 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of an amphiphilic agent in a composition described herein for a 24-hour period may be about 0 μg to about 10 g, about 0.01 g to about 4 g, about 0.01 g to about 2.0 g, about 0.01 g to about 1.0 g, about 0.01 g to about 0.5 g, about 0.01 g to about 0.1 g, about 0.01 g to about 0.05 g, about 0.01 g to about 0.04 g, or about 0.01 g to about 0.03 g, or more and values therebetween. For example, the total dosage of an amphiphilic agent in a composition for a 24-hour period may be about 0μ, 10μ, 15μ, 20μ, 25μ, 30μ, 35μ, 40μ, 45μ, 50μ, 55μ, 60μ, 65μ, 70 μg, 75 μg, 80 μg, 85 μg, 90 μg, 95 μg, 100 μg, 125 μg, 150 μg, 175 μg, 200 μg, 225 μg, 250 μg, 275μ, 300μ, 325μ, 350μ, 375μ, 400μ, 425μ, 450μ, 475μ, 500μ, 525μ, 575μ, 600μ g, 625μ, 650μ, 675μ, 700μ, 725μ, 750μ, 775μ, 800μ, 825μ, 850μ, 875μ, 900μ g, 925 μg, 950 μg, 975 μg, 1000 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of a stimulant in a composition described herein for a 24-hour period may be about 0 μg to about 10 g, about 0.001 g to about 4 g, about 0.001 g to about 2.0 g, about 0.001 g to about 1.0 g, about 0.001 g to about 0.5 g, about 0.001 g to about 0.1 g, about 0.001 g to about 0.05 g, about 0.001 g to about 0.04 g, about 0.001 g to about 0.03 g, about 0.002 g to 0.01 g, about 0.003 g to about 0.008 g, about 0.004 g to about 0.006 g, or any values therebetween. For example, the total dosage of a stimulant in a composition for a 24-hour period may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 ug, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of HMB in a composition described herein for a 24-hour period may be about 0 g to about 10 g, about 0.1 g to about 6.0 g, about 0.2 g to about 4.0 g, about 0.5 g to about 3.0 g, about 1.0 g to about 2.0 g, or more and any values therebetween. For example, the total dosage of HMB in a composition for a 24-hour period may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

A composition described herein can be given on a daily basis over a period of about 1 day to about four or more weeks. In certain embodiments, the total dosage of beta-alanine in a composition for each day may be about 0.01 g to about 10 g, about 0.02 g to about 6.4 g, about 0.04 g to about 3.2 g, about 0.08 g to about 2.4 g, about 0.1 g to about 2 g, about 0.2 g to about 1.8 g, about 0.3 g to about 0.6 g, about 1.0 g to about 1.8 g, about 0.1 g, about 0.2 g, about 0.4 g, about 0.54 g, about 0.84 g, about 1.12 g, about 1.2 g, about 1.6 g, about 1.68 g, about 2.34 g, about 2.4 g, about 3.2 g, about 3.36 g, about 4.68 g, about 4.8 g, or about 6.4 g, and values therebetween. In certain embodiments, a composition contains about 0.1 g, 0.15 g, 0.2 g, 0.25 g, 0.27 g, 0.3 g, 0.35 g, 0.4 g, 0.45 g, 0.5 g, 0.55 g, 0.6 g, 0.65 g, 0.7 g, 0.75 g, 0.8 g, 0.84 g, 1.12 g, 1.2 g, 1.6 g, 1.68 g, 2.34 g, 2.4 g, 3.2 g, 3.36 g, 4.68 g, 4.8 g, or 6.4 g, and values therebetween, of beta-alanine. In certain embodiments, the total dosage of an anti-inflammatory agent in a composition for each day may be about 0.001 g to about 1 g, about 0.002 g to about 0.5 g, about 0.002 g to about 0.004 g, about 0.004 g to about 0.008 mg, or about 0.002 g to about 0.25 g, and values therebetween. In certain embodiments, the total dosage of an amphiphilic agent in the composition for each day may be about 0.001 g to about 1 g, about 0.002 g to about 0.5 g, about 0.005 g to about 0.25 g, about 0.012 g to about 0.024 g, or about 0.008 g to about 0.1 g, and values therebetween. The amount of a composition administered can be adjusted daily, weekly, or monthly. In certain embodiments, the amount of a composition administered can be increased daily, every two days, every three days, every four days, every five days, and every six days. In certain embodiments, the amount of a composition administered can be increased weekly, every two weeks, every three weeks, every four weeks, every five weeks, and every six weeks. In certain embodiments, the amount of a composition administered can be increased daily for about three days, four days, five days, six days, seven days, fourteen days, twenty-one days, twenty-eight days, thirty days, sixty days, or more or any days in between followed by a constant dosage, a decreased dosage, or a combination thereof. In certain embodiments, a composition that does not contain an anti-inflammatory agent may be administered first followed by administration of an anti-inflammatory agent-containing composition, where the interval between the two administrations can be from about 15 minutes to 24 hours. In certain embodiments, a composition that does not contain an anti-inflammatory agent and an anti-inflammatory agent-containing composition can be administered alternately. In certain embodiments, the amount of a composition administered can be decreased daily, every two days, every three days, every four days, every five days, and every six days. In certain embodiments, the amount of a composition administered can be decreased weekly.

Compositions described herein may be administered alone or in conjunction with other supplements or drugs useful in improving cognitive function.

In some embodiments, an unencapsulated beta-alanine composition may be administered to a subject for a therapeutic or nutraceutical outcome (as described herein) that is different than the therapeutic or nutraceutical outcome that an encapsulated beta-alanine composition is administered to a subject for. In certain embodiments, an encapsulated beta-alanine composition may be administered to a subject for the same purpose as an unencapsulated beta-alanine composition. Each form of beta-alanine can be administered at the frequencies described herein. Compositions described herein may be administered as the sole treatment or in conjunction with other supplements, vitamins, or drugs useful for any of the outcomes described herein.

Certain embodiments demonstrate unexpectedly improved stability, without being bound by any particular theory, due to improved resistance to moisture absorption. Certain embodiments comprise a composition possessing unexpectedly improved stability (e.g., shelf life) wherein the composition remains stable for about 1 month, 2 months, 3 months, 6 months, 12 months, 18 months, 24 months, 36 months, or more and values therebetween. Certain embodiments comprise a beta-alanine composition wherein the composition has unexpectedly improved bioavailability compared to prior art beta-alanine compositions wherein the composition can be administered orally and achieve the improved bioavailability.

Certain embodiments comprise a beta-alanine composition wherein the beta-alanine composition provides unexpectedly superior suppression of lactate and lactic acid buildup in the muscle, for example, during anaerobic and/or aerobic exercise. Across a range of subjects, certain embodiments, as described herein, comprise a beta-alanine composition wherein the composition provides a more uniform reduction in lactate concentration in subjects compared to prior art beta-alanine compositions. Certain embodiments comprise a beta-alanine composition as described herein wherein the beta-alanine composition is administered to effectively reduce cortisol concentration and/or rate of production in a subject. Certain embodiments comprise a beta-alanine composition as described herein wherein the beta-alanine composition is administered to effectively reduce C-reactive protein concentration and/or rate of production in a subject. Certain embodiments comprise a beta-alanine composition, as described herein, wherein the beta-alanine composition is administered to a subject to increase carnosine production and/or concentration in a subject's tissues, wherein the tissues are selected from the group consisting of skeletal muscle, brain tissue, heart muscle, cells, and combinations thereof.

Certain embodiments comprise a beta-alanine composition, as described herein, wherein the composition is administered to improve and/or maintain a healthy mood in a subject. For example and without limitation, indicators of a healthy and/or improved mood include one or more of tenseness, anger, fatigue (e.g. “worn out”), happiness, pride, liveliness, confusion, sadness, activeness, feelings of being “on-edge,” grouchiness, shame, energy, hopelessness, uneasiness, restlessness, concentration and/or focus, fatigue, competency, annoyance, discouragement, resentfulness, nervousness, feelings of being miserable, confidence, bitterness, exhaustion, anxiety, helplessness, weariness, satisfaction, bewilderment, fury, enthusiasm, worthlessness, forgetfulness, vigor, uncertainty, embarrassment, and the like.

Certain embodiments comprise a beta-alanine composition, as described herein, wherein the beta-alanine composition provides unexpectedly superior improvements in inflammatory markers, e.g. cytokines, interleukins (e.g., IL-6 and IL-10 and the like), and the like, compared to prior art beta-alanine compositions.

Certain embodiments comprise a beta-alanine composition, as described herein, wherein the composition provides unexpectedly superior improves in physical endurance and athletic performance. Without being bound by any particular theory, compositions as described herein can provide unexpectedly improved gains in short-term muscle exertion (e.g., weight lifting, sprinting, muscle twitch speed, and the like) and/or unexpectedly improved gains in long-term muscle exertion (e.g., long-distance running, aerobic activity, swimming, long-distance cycling, and the like).

A method of preparing a composition according to the disclosure contained herein, is exemplified by the following steps:
step (a) adding an amphiphilic agent in water to form a first solution;
step (b) homogenizing the first solution to form a first emulsion;
step (c) adding an anti-inflammatory agent into the first emulsion to form a second solution;
step (d) homogenizing the second solution to form a second emulsion;
step (e) adding beta-alanine into the second emulsion to form a third solution;
step (f) homogenizing the third solution to form a third emulsion;
step (g) adding a stimulant into the third emulsion to form a fourth solution;
step (h) homogenizing the fourth solution to form a fourth emulsion;
step (i) adding an aqueous solution of beta-cyclodextrin into the third emulsion to form a fifth solution;
step (j) homogenizing the fifth solution to form a fifth emulsion;
step (k) nano milling the fifth emulsion; and
step (l) spray-drying the nano-milled emulsion to form a dried beta-alanine composition.

A beta-alanine composition can also be prepared by the following steps:
step (a) adding an amphiphilic agent in water to form a first solution;
step (b) homogenizing the first solution to form a first emulsion;
step (c) adding a mixture of an anti-inflammatory agent, beta-alanine and a stimulant into the first emulsion to form a second solution;
step (d) homogenizing the second solution to form a second emulsion;
step (e) adding an aqueous solution of beta-cyclodextrin into the second emulsion to form a third solution;
step (f) homogenizing the third solution to form a third emulsion;
step (g) nano milling the third emulsion; and
step (h) spray-drying the third emulsion to form a beta-alanine composition.

In certain embodiments, when a composition does not contain an ingredient such as a stimulant, the steps involving such an ingredient can be omitted. The disclosure contained herein contemplates that the foregoing method steps can be rearranged as would be envisaged by the skilled artisan in view of the disclosure and claims set forth herein.

In certain embodiments, methods described herein further comprise mixing HMB with a beta-alanine composition as described herein.

The terminology used herein is for the purpose of describing particular embodiments only and is not intended to be limiting. As used in the Specification and the appended claims, the singular forms “a”, “an” and “the” include plural referents unless the context clearly dictates otherwise.

The terms “reducing” is used herein to generally refer to relieving the disease, causing regression of the disease and/or its symptoms, conditions, and co-morbidities and obtaining a desired pharmacological and physiological effect and can also refer to a nutritional or nutraceutical effect, the scopes, and meanings of which will be clear to the skilled artisan based upon the context in which these terms are used. As set forth herein, any composition that is administered to reduce any condition, can also be administered to maintain a healthy level of a physiological or biological condition. In certain embodiments, a composition is administered to maintain a healthy level of one or more of the conditions disclosed herein. The scope and meaning of “reducing” and “maintaining healthy levels of” would be immediately envisaged by the skilled artisan when viewing the term in the context of the disclosure and the claims.

The term “liposomal” and “liposome” described herein can refer to an artificial vesicle having at least one lipid bilayer. The vesicle can be multilamellar large, oligolamellar, small unilamellar, medium-sized unilamellar, large unilamellar, giant unilamellar, and multivesicular.

The term “pharmaceutical composition”, “composition” and the like can refer to preparations which are in such a form as to permit the biological activity of the active ingredients to be effective, and therefore may be administered to a subject for therapeutic use along with dietary and/or nutritional supplement use. The term “emulsion” can refer to a mixture of two or more liquids that are normally immiscible owing to liquid-liquid separation. The term “homogenize” can refer to any processes used to make a mixture of two mutually non-soluble liquids the same throughout by turning one of the liquids into a state consisting of extremely small particles distributed uniformly throughout the other liquid. The term “spray-drying” can refer to a process involving the atomization of a solution, slurry, or emulsion containing one or more components of the desired product into droplets by spraying followed by the rapid evaporation of the sprayed droplets into solid powder by hot air at a certain temperature and pressure. The meaning of these terms will be clear to the skilled artisan based upon the context in which they are used.

As provided herein, the disclosure of a “ratio” of compounds and compositions corresponds to a ratio provided in terms of weight mass of the components present in the ratio.

As used herein, the term “subject” refers to animals, including mammals and humans.

As used herein, the terms “attenuating the aging process” and “anti-aging effects” can be used interchangeably.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

### EXAMPLES

### Example 1: Preparation of a Beta-Alanine Composition

Lecithin (2.5 g) was dissolved in water (250 mL) at 70 °C and homogenized for 10 minutes to form an emulsion. To the emulsion, beta-caryophyllene (2.94 g, i.e., 98%) in clove oil (3.0 g) was added. The resultant solution was homogenized for 10 minutes, then beta-alanine (86 g) was added to form a solution. The resultant solution was homogenized for 15 minutes and then caffeine (0.22 g) was added. The resultant solution was homogenized for 15 minutes, followed by adding a water solution (150 mL) of beta cyclodextrin (8.08 g). The resultant mixture was nano-milled for 20 minutes and then spray dried using an outlet temperature of 90 °C and inlet temperature of 180 °C to provide a dry powder. The powder (85 g) was blended homogeneously with a mixture of flavor agents (12 g) and sucrose powder (3 g) for 30 minutes to give a beta-alanine composition (Composition 1).

### Example 2: Preparation of a Composition Further Comprising HMB

Composition 1 (600 mg) was further blended with HMB-FA (1.5 g) to form Composition 2.

### Example 3: Permeability Study

The bioavailability of Compositions 1 and 2 was evaluated by an in vitro permeability study using a Caco-2 permeability assay and were compared to free unencapsulated beta-alanine (“Reference product 1”) and a formulated beta-alanine composition containing beta-alanine, beta-caryophyllene, lecithin, caffeine, modified food starch, flavor agents and sucrose powder (“Reference product 2”).

The Caco-2 cell line is frequently used to examine intestinal permeability, determine drug absorption, and investigate passive and active diffusion transport of drug molecules. Transported test substances passed through the Caco-2 monolayer intact and non-metabolized can be detected by LC-MS in the basolateral or apical samples taken after the given time point exposure at the selected concentration of a sample across Caco-2 cell monolayer.

### Cell Line and Culture Medium

Caco-2 cells (from National Centre for Cell Sciences, Pune, India) were cultured in a 25 cm² tissue culture flask with Minimum Essential Medium (MEM; from Sigma Aldrich, USA) supplemented with 20% Fetal Bovine Serum (FBS) and antibiotic solution containing Penicillin (100 U/ml), Streptomycin (100 μg/ml), and Amphotericin B (2.5 μg/ml) at 37 °C in a humidified 5% CO₂ incubator (NBS Eppendorf, Germany). The viability of cells was evaluated by direct observation of cells by inverted phase contrast microscope and followed by MTT assay method.

### Cells Seeding in a 96-Well Plate

Two days old confluent monolayer of cells were trypsinized and the cells were suspended in 10% growth medium, and 100 μL cell suspension (5×10³ cells/well) was seeded in a 96-well tissue culture plate and incubated at 37 °C in a humidified 5% CO₂ incubator.

### Preparation of Sample Solutions

1 mg of each of Reference product 1, Reference product 2, Composition 1 and Composition 2 was weighed and dissolved in 1 mL 0.1% DMSO using a cyclomixer. The resultant solutions were filtered through 0.22 μm Millipore syringe filter to ensure the sterility.

### Cytotoxicity Study

This study was conducted to ensure the accuracy of cell permeability results; namely, increased cell permeability was not caused by damages of cells.

### Cytotoxicity Study by Direct Microscopic Observation

After 24 hours of the growth, medium was removed, freshly prepared each sample solution was added to the cells at concentrations of 100 μg/mL, 50 μg/mL, 25 μg/mL, 12.5 μg/mL, 6.25 μg/mL. Each composition at each concentration was added in triplicates to the respective wells and incubated at 37 °C in a humidified 5% CO₂ incubator. Untreated control cells were also maintained. Entire plate was observed after 72 hours of treatment using an inverted phase contrast tissue culture microscope (Olympus CKX41 with Optika Pro5 CCD camera) and microscopic observations were recorded as images. Any detectable changes in the morphology of the cells, such as rounding or shrinking of cells, granulation and vacuolization in the cytoplasm of the cells were considered as indicators of cytotoxicity. No detectable changes were observed for all the four compositions, indicating that they are not toxic even at a high concentration of 100 μg/mL.

### Cytotoxicity Study Using MTT Method

Reference product 1, Reference product 2, Composition 1 and Composition 2 were also evaluated for their in vitro cytotoxicity against Caco-2 cells by using the 3-(4,5-dimethylthiazol-2-yl)-2,5-diphenyl-2H-tetrazolium bromide (MTT) assay in semi-confluent monolayer cultures at concentrations of 100 μg/mL, 50 μg/mL, 25 μg/mL, 12.5 μg/mL, 6.25 μg/mL. 15 mg of MTT (Sigma, M-5655) was reconstituted in 3 mL PBS until completely dissolved and sterilized by filter sterilization. After 72 hours of incubation period, the sample contents in wells were removed and 30 μL of reconstituted MTT solution was added to all test and control wells, the plate was gently shaken well, then incubated at 37 °C in a humidified 5% CO₂ incubator for 4 hours. After the incubation period, the supernatant was removed and 100 μL of MTT solubilization dimethyl sulfoxide (DMSO; Sigma Aldrich, USA) was added, and the wells were mixed gently by pipetting up and down to solubilize the formazan crystals. The absorbance values were measured by using microplate reader at a wavelength of 540 nm. Cell viability was calculated using the following formula:

Viability (%)=100×(Mean sample/Mean control group).

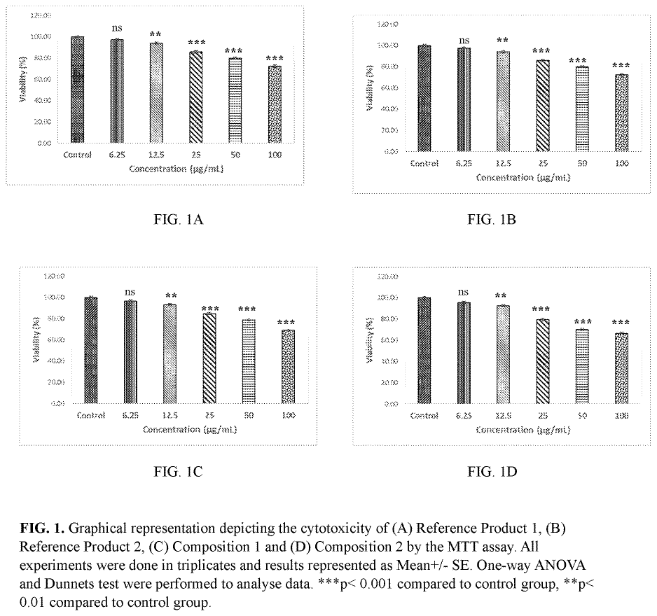

The dose-response cell viability of each composition is shown in FIGS. 1A-1D. The results show that Reference product 1, Reference product 2, Composition 1 and Composition 2 all exhibited low cytotoxicity, indicating that they are safe even at a high concentration of 100 μg/mL. The IC₅₀ values calculated by using ED50 PLUS V1.0 Software of Reference product 1, Reference product 2, Composition 1 and Composition 2 are respectively 202.030 μg/mL, 179.0712 μg/mL, 160.6939 μg/mL and 140.6225 μg/mL, also indicating their low cytotoxicity.

### Caco-2 Cell Permeability Study

Caco-2 cells of passage 31 were seeded on polyester membrane inserts (6.5 mm, 0.33 cm², 0.4 μm pore size) at a density of 100,000 cells per insert and cultured at 37 °C for 25 days for the intestinal permeability tests, where culture medium was changed three times a week to allow the formation of a confluent monolayer. The integrity of the monolayer was assessed by the permeability to the paracellular marker Lucifer Yellow (LY) dye.

The growth medium was removed and 1 mL prewarmed Hank's Balanced Salt Solution (HBSS) (pH 7.4) was added to the basolateral chamber. To the apical chamber, 20 μM LY solution was added and incubated at 37 °C for 30 minutes. After 30 minutes 30 μL of sample from an apical chamber and 500 μL of sample from the basolateral chamber were collected and fluorescence were read using a Qubit fluorimeter (excitation 428 nm, emission 536 nm). The apparent permeability coefficient (Papp) was calculated based on the formula below:

$${P}_{app} ( cm / min ) = \frac{{V}_{B}}{A \times {C}_{A 0}} \times \frac{Δ {C}_{B}}{Δ t}$$

V_{B} is the volume in the basolateral chamber, A is the surface area of the transwell insert (0.33 cm²), C_{A0} is the initial concentration in the apical compartment, and ΔC_{B}/Δt is the change in concentration over time in the basolateral compartment.

Inserts with permeability coefficient ranging from 2.3×10⁻⁴ and 2.8×10⁻⁴ (cm/min) and more than 80% LY recovery were used further for the permeability study.

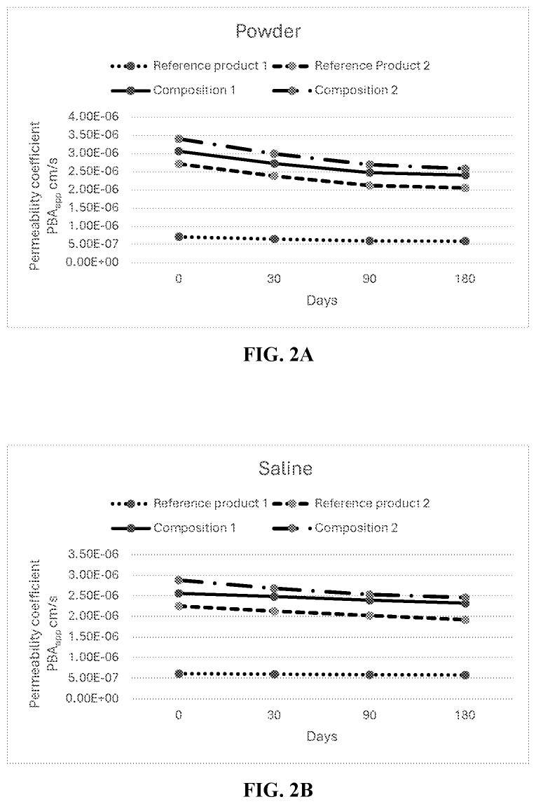

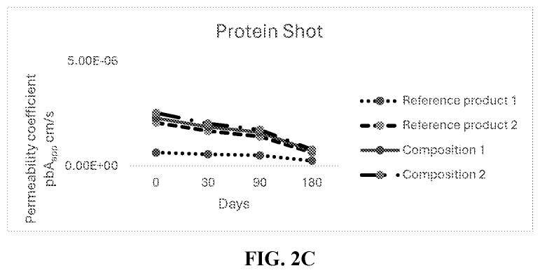

To mimic in vivo conditions, HBSS was used as transport buffer in the apical (AP) and basolateral compartments (BL), respectively at pH 6.0 and pH 7.4. After determining the membrane integrity with LY, the donor compartment received DMSO stock solutions of each of Reference product 1 at a concentration of 25 μg/mL, Reference product 2 at a concentration of 25 μg/mL, Composition 1 at a concentration of 25 μg/mL and Composition 2 at a concentration of 12.5 g/mL. Reference product 1, Reference product 2, Composition 1 and Composition 2 were stored as powders, in saline solutions with a salt concentration of about 1%, and in protein shot solutions (containing about 27 wt % of marine collagen and about 18 wt % of whey protein) in accelerated stability chambers. 180 days of accelerated stability testing are scientifically and regulatory accepted as equivalent to 18 months of real-time shelf life based on the ICH guidelines and the industry standards. Samples stored at day 0, day 30, day 90, and day 180 were taken and then diluted with HBSS. The receiver compartment received freshly prepared HBSS. All experiments were carried out in a single direction, from AP to BL, for the required 120 minutes at 37 °C, and with an orbital shaker spinning at 100 rpm. After 120 minutes, 100 μL aliquots from the receiver compartments were taken out to determine the penetration coefficient of the marker molecules in Caco2 cells. Samples from donor compartments were taken at the end of experiment. Sample concentrations were assessed in both chambers using validated LC-MS method. The following formula was used to calculate the permeability coefficient (PBAₐₚₚ):

$${PBA}_{app} = \frac{Δ Q}{Δ T} \times \frac{1}{A * {C}_{0} * 60}$$

where ΔQ/ΔT is the steady state flux of beta-alanine (mol/sec), A is the surface area of the filter (mm²), and C₀ is the initial concentration of beta-alanine in the each of Reference product 1, Reference product 2, Composition 1 and Composition 2 applied in the apical layer (mol/ml). The results are shown in FIGS. 2A-2C and Tables 1-3 below.

TABLE 1
PBAₐₚₚ of the test samples as powders

| | PBAₐₚₚ Apical to Basolateral (cm/s) | | | | |
|---|---|---|---|---|---|
| Test sample | Day 0 | Day 30 | Day 90 | Day 180 | Average |
| Reference product 1 | 7.07 × 10⁻⁷ | 6.45 × 10⁻⁷* | 5.95 × 10⁻⁷* | 5.89 × 10⁻⁷* | 6.34 × 10⁻⁷ |
| Reference product 2 | 2.71 × 10⁻⁶ | 2.38 × 10⁻⁶* | 2.12 × 10⁻⁶* | 2.05 × 10⁻⁶* | 2.32 × 10⁻⁶ |
| Composition 1 | 3.06 × 10⁻⁶ | 2.72 × 10⁻⁶* | 2.47 × 10⁻⁶* | 2.40 × 10⁻⁶* | 2.66 × 10⁻⁶ |
| Composition 2 | 3.40 × 10⁻⁶ | 2.99 × 10⁻⁶* | 2.69 × 10⁻⁶* | 2.58 × 10⁻⁶* | 2.92 × 10⁻⁶ |

Paired t-test was performed to analyse data.
*Statistically significant at p < 0.05 level.

TABLE 2
PBAₐₚₚ of the test samples in saline solutions

| | PBAₐₚₚ Apical to Basolateral (cm/s) | | | | |
|---|---|---|---|---|---|
| Test sample | Day 0 | Day 30 | Day 90 | Day 180 | Average |
| Reference product 1 | 6.12 × 10⁻⁷ | 6.00 × 10⁻⁷* | 5.84 × 10⁻⁷* | 5.78 × 10⁻⁷* | 5.93 × 10⁻⁷ |
| Reference product 2 | 2.25 × 10⁻⁶ | 2.13 × 10⁻⁶* | 2.02 × 10⁻⁶* | 1.92 × 10⁻⁶* | 2.08 × 10⁻⁶ |
| Composition 1 | 2.56 × 10⁻⁶ | 2.48 × 10⁻⁶* | 2.39 × 10⁻⁶* | 2.32 × 10⁻⁶* | 2.44 × 10⁻⁶ |
| Composition 2 | 2.88 × 10⁻⁶ | 2.68 × 10⁻⁶* | 2.53 × 10⁻⁶* | 2.46 × 10⁻⁶* | 2.64 × 10⁻⁶ |

Paired t-test was performed to analyse data.
*Statistically significant at p < 0.05 level.

TABLE 3
PBAₐₚₚ of the test samples in protein shot solutions

| | PBAₐₚₚ Apical to Basolateral (cm/s) | | | | |
|---|---|---|---|---|---|
| Test sample | Day 0 | Day 30 | Day 90 | Day 180 | Average |
| Reference product 1 | 6.51 × 10⁻⁷ | 5.67 × 10⁻⁷* | 5.16 × 10⁻⁷* | 2.53 × 10⁻⁷* | 4.97 × 10⁻⁷ |
| Reference product 2 | 2.07 × 10⁻⁶ | 1.68 × 10⁻⁶* | 1.41 × 10⁻⁶* | 6.51 × 10⁻⁷* | 1.45 × 10⁻⁶ |
| Composition 1 | 2.29 × 10⁻⁶ | 1.87 × 10⁻⁶* | 1.61 × 10⁻⁶* | 7.30 × 10⁻⁷* | 1.63 × 10⁻⁶ |
| Composition 2 | 2.54 × 10⁻⁶ | 2.03 × 10⁻⁶* | 1.74 × 10⁻⁶* | 7.86 × 10⁻⁷* | 1.74 × 10⁻⁶ |

Paired t-test was performed to analyse data.
*Statistically significant at p < 0.05 level.

FIG. 2A and Table 1 show that permeability of all the test compositions as powders change very little over 180 days, indicating that they are stable over 180 days in accelerated stability chambers and thus have at least 18 months of shelf life. FIG. 2A and Table 1 also show that (i) Composition 1 and Composition 2 as powders each unexpectedly exhibited at least three-fold higher permeability than Reference product 1 and (ii) the Composition 2 powder unexpectedly exhibited at least two-fold higher permeability than the Reference product 2 powder given the fact the concentration of the former was only half of that of the latter.

FIG. 2B and Table 2 show that permeability of all the test compositions in saline solutions change very little over 180 days, indicating that they are stable over 180 days in accelerated stability chambers and thus have at least 18 months of shelf life. FIG. 2B and Table 2 also show that (i) Composition 1 and Composition 2 in saline solutions each unexpectedly exhibited at least three-fold higher permeability than Reference product 1 and (ii) the Composition 2 saline solution unexpectedly exhibited at least two-fold higher permeability than the Reference product 2 saline solution given the fact that the concentration of the former was only half of that of the latter. The results demonstrate that compositions described herein can be added into water-based beverages, such as flavored water, sport drinks, energy drinks, saline drinks, and juice, and the resultant aqueous mixtures are stable (having at least 180 days of shelf life) and thus provide a convenient way for people to supplement beta-alanine and allow for premade (i.e., “ready-to-drink”) beverages to include a beta-alanine composition with improved bioavailability that also possesses an improved shelf-life for such products, which was not achievable with prior art compositions.

FIG. 2C and Table 3 show that Composition 1 and Composition 2 formulated as protein shot solutions throughout the 180 days unexpectedly exhibited higher permeability than Reference product 1 formulated as protein shot solutions. The results also indicate that compositions described herein can be taken concurrently with protein shots.

Because higher permeability is associated with better bioavailability, the results indicate, in conjunction with the other aspects described herein, that compositions described herein would be unexpectedly more bioavailable than conventional beta-alanine and other forms of beta-alanine disclosed prior to the present invention.

The integrity of the monolayers was assessed at the end of the study to ensure that the differences in permeability were not caused by a loss in monolayer integrity. The monolayers were washed twice with HBSS at pH 7.4 and incubated for 30 minutes at 37 °C. HBSS was then added into the apical layer and each solution was added individually to the basolateral layer. It was observed that the integrity of the monolayers treated with each sample remained unchanged.

### Example 4: Oral Bioavailability Study

### Oral Bioavailability Study

The oral bioavailability of Composition 1 described herein was evaluated in a double-blind, randomized, single-dose, three-treatment, three-way crossover study in healthy human adults under fasting conditions as compared to Reference product 1 and Reference product 2. Twelve healthy human adults (age of 50-65 years) were enrolled in the study and randomly divided into three treatment groups with four participants in each group in a sequential manner. Group 1 was initially dosed orally with Composition 1 (containing 400 mg of beta-alanine), Group 2 was initially dosed orally with Reference product 1 (containing 400 mg of conventional beta-alanine) and Group 3 was initially dosed orally with Reference product 2 (containing 400 mg of beta-alanine). After an interim washout period of seven days, Group 1 was dosed with Reference product 1, Group 2 was dosed with Reference product 2, and Group 3 was dosed with Composition 1. After another interim washout period of seven days, Group 1 was dosed with Reference product 2, Group 2 was dosed with Composition 1, and Group 3 was dosed with Reference product 1. At the end of the study, each participant was dosed with Composition 1, Reference product 1, and Reference product 2 (i.e., each participant had three treatments). Composition 1, Reference product 1, and Reference product 2 each was administered as a liquid dosage form which was prepared 30 days prior to the study by mixing each of them individually with 250 ml of water and sealed in individual containers. All the participants were fasted for at least eleven hours before each dosing. In the morning and within one hour before dosing, a 5 mL blood sample from each participant's forearm vein was collected. Post-dose blood samples from each participant's forearm vein was collected at hour 0.25, hour 0.5, hour 1, hour 2, hour 4, hour 6, and hour 8 (5 mL each time; 8 blood samples from each participant) and placed in vacutainers containing K₂EDTA. An indwelling cannula was used to draw the blood and heparin-lock technique will be used to prevent blood from clotting in the indwelling cannula. Before drawing each blood sample drawn via the indwelling cannula, 0.5 mL of blood was discarded to prevent the saline diluted blood and heparin (10 IU/mL) from interfering with the analysis. The vacutainers was placed upright in a rack in a wet ice bath until centrifugation and during separation. At hour 5 after dosing, each participant was provided with a standardized vegan meal free from beta-alanine and L-histidine (including fresh salads, grilled vegetables dressed with vegetable oils, egg-free mayonnaise, dried spices and fruit). The collected blood samples were placed in a thermos-insulated box containing wet ice and the box will be transferred to the sample processing room. Within 30 minutes of each blood sample collection, the blood sample was centrifuged at 4000±50 RPM for 10 minutes at 2 °C-8 °C to separate the plasma. The separated plasma was equally transferred to polypropylene tube in two aliquots which were stored upright in a freezer at −30 °C±10 °C or −70 °C±15 °C at a clinic and analytical site. The plasma samples were transferred to an analytical site after the completion of the clinical phase. Beta-alanine concentrations in the plasma samples were assessed using a validated analytical method. Statistical analyses were performed to determine pharmacokinetic (PK) parameters Cₘₐₓ (μmol/L), Kₑₗ, t_{1/2} (h), AUC₀₋ₜ (μmol·h/L), AUC_{0-∞} (μmol·h/mL), and Tₘₐₓ (h) using SAS® system for windows version 9.4 or above (SAS® Institute Inc., USA). The mean plasma beta-alanine concentrations are shown in Table 4 and the average PK variables (mean±SD) are shown in Table 5.

TABLE 4

| Time | Composition 1 | Reference Product 2 | Reference Product 1 |
|---|---|---|---|
| (h) | (μmol/L) | (μmol/L) | (μmol/L) |
| 0.00 | 0.00 ± 0.00 | 0.00 ± 0.00 | 0.00 ± 0.00 |
| 0.25 | 11.53 ± 1.88 | 13.03 ± 2.18 | 4.07 ± 1.01 |
| 0.50 | 29.52 ± 5.60 | 32.50 ± 6.06 | 10.99 ± 1.90 |
| 1.00 | 62.03 ± 6.03 | 65.77 ± 5.66 | 23.43 ± 2.58 |
| 2.00 | 106.75 ± 10.45 | 112.33 ± 10.38 | 39.03 ± 3.14 |
| 4.00 | 88.25 ± 9.29 | 93.70 ± 7.22 | 13.44 ± 2.15 |
| 6.00 | 48.05 ± 6.34 | 52.42 ± 8.06 | 6.26 ± 1.06 |
| 8.00 | 17.03 ± 1.78 | 20.39 ± 2.33 | 3.50 ± 0.35 |

TABLE 5

| PK parameters | Composition 1 | Reference Product 2 | Reference Product 1 | P values |
|---|---|---|---|---|
| Cₘₐₓ (μmol/L) | 106.75 ± 10.45 | 112.33 ± 10.38 | 39.03 ± 3.14 | <0.01* |
| AUC₀₋ₜ = AUC_{0-8 h} | 510.21 ± 72.48 | 545.89 ± 76.67 | 124.16 ± 18.69 | <0.001* |
| (μmol · h/L) | | | | |
| Tₘₐₓ (h) | 2.50 ± 0.90 | 2.00 ± 0.00 | 2.00 ± 0.00 | <0.05* |
| t_{1/2} (h) | 2.33 ± 0.38 | 2.43 ± 0.46 | 1.87 ± 0.37 | <0.01* |
| Kₑₗ | 0.31 ± 0.05 | 0.30 ± 0.06 | 0.38 ± 0.07 | <0.01* |
| AUC_{0-∞} | 567.46 ± 26.23 | 617.79 ± 42.17 | 133.62 ± 9.58 | <0.001* |
| (μmol · h/mL) | | | | |

Comparison between the study groups using ANOVA with the level of significance (α) as P < 0.05.
*Statistically significant.

The results show that Composition 1 has similar bioavailability to Reference Product 2 and both Composition 1 and Reference Product 2 exhibited unexpectedly much higher bioavailability than Reference Product 1.

### Example 5: Paraesthesia Sensation Evaluation

Compositions 1 and 2 were evaluated for their paraesthesia sensation.

Five healthy subjects were dosed with Reference Product 1 (as control) in distilled water (600 mg beta-alanine/40 mL) for 7 days, Reference Product 1 in a protein shot (600 mg beta-alanine+20 g protein/40 mL), Reference Product 1 plus HMB in a protein shot (600 mg beta-alanine+1.5 g HMB/40 mL), Composition 1 in distilled water (600 mg/40 mL) and Composition 2 in a protein shot (600 mg Composition 1+1.5 g HMB+20 g protein/40 mL). Each subject scaled their paraesthesia sensation, with the score of 10 indicating no paraesthesia and the score of 0 indicating severe paraesthesia.

As shown in Table 6 below, compositions described herein exhibited significantly less degree of paraesthesia sensation than Reference Product 1, which is an unexpected advantage over a prior art product for their applications in aqueous beverages and nutritional beverages.

TABLE 6

| | | Itching | Itching | Tingling | Total | | |
|---|---|---|---|---|---|---|---|
| Pannel | | effect | effect | effect | Mouth | After | Mean |
| member | Product | on Skin | on Throat | on body | feel | taste | Score |
| Pannel | Control in Distilled Water | 5 | 5 | 5 | 5 | 5 | 5 |
| Member | Control in Protein Shot | 6 | 6 | 6 | 6 | 5 | 6 |
| 1 | Control + HMB in Protein | 6 | 6 | 4 | 6 | 6 | 5.5 |
| | Shot | | | | | | |
| | Composition 1 in | 8 | 8 | 8 | 8 | 8 | 8 |
| | Distilled Water | | | | | | |
| | Composition 1 in Protein | 9 | 9 | 9 | 9 | 9 | 9 |
| | Shot | | | | | | |
| | Composition 2 in Protein | 9 | 9 | 9 | 9 | 9 | 9 |
| | Shot | | | | | | |
| Pannel | Control in Distilled Water | 4 | 5 | 4 | 4 | 4 | 4.25 |
| Member | Control in Protein Shot | 4 | 4 | 4 | 4 | 4 | 4 |
| 2 | Control + HMB in Protein | 5 | 4 | 4 | 4 | 4 | 4.25 |
| | Shot | | | | | | |
| | Composition 1 in | 8 | 7 | 7 | 9 | 9 | 7.75 |
| | Distilled Water | | | | | | |
| | Composition 1 in Protein | 9 | 8 | 8 | 9 | 9 | 8.5 |
| | Shot | | | | | | |
| | Composition 2 in Protein | 10 | 8 | 8 | 8 | 8 | 8.5 |
| | Shot | | | | | | |
| Pannel | Control in Distilled Water | 4 | 4 | 4 | 4 | 4 | 4 |
| Member | Control in Protein Shot | 6 | 5 | 5 | 5 | 5 | 5.25 |
| 3 | Control + HMB in Protein | 5 | 6 | 5 | 5 | 5 | 5.25 |
| | Shot | | | | | | |
| | Composition 1 in | 8 | 8 | 8 | 8 | 8 | 8 |
| | Distilled Water | | | | | | |
| | Composition 1 in Protein | 9 | 10 | 9 | 9 | 9 | 9.25 |
| | Shot | | | | | | |
| | Composition 2 in Protein | 9 | 9 | 9 | 9 | 9 | 9 |
| | Shot | | | | | | |
| Pannel | Control in Distilled Water | 4 | 4 | 4 | 4 | 4 | 4 |
| Member | Control in Protein Shot | 6 | 5 | 5 | 6 | 6 | 5.5 |
| 4 | Control + HMB in Protein | 5 | 6 | 5 | 5 | 5 | 5.25 |
| | Shot | | | | | | |
| | Composition 1 in | 7 | 8 | 8 | 7 | 7 | 7.5 |
| | Distilled Water | | | | | | |
| | Composition 1 in Protein | 9 | 9 | 9 | 9 | 9 | 9 |
| | Shot | | | | | | |
| | Composition 2 in Protein | 9 | 9 | 10 | 9 | 9 | 9.25 |
| | Shot | | | | | | |
| Pannel | Control in Distilled Water | 4 | 4 | 4 | 4 | 4 | 4 |
| Member | Control in Protein Shot | 4 | 5 | 5 | 5 | 5 | 4.75 |
| 5 | Control + HMB in Protein | 5 | 5 | 5 | 5 | 5 | 5 |
| | Shot | | | | | | |
| | Composition 1 in | 7 | 7 | 8 | 8 | 8 | 7.5 |
| | Distilled Water | | | | | | |
| | Composition 1 in Protein | 9 | 9 | 9 | 9 | 9 | 9 |
| | Shot | | | | | | |
| | Composition 2 in Protein | 9 | 9.5 | 9 | 9 | 9 | 9.125 |
| | Shot | | | | | | |

### Example 6: Stability of Compositions

Composition 1 was evaluated for its stability under both long-term and accelerated storage conditions relevant to routine handling and storage. The study was designed in accordance with the principles outlined in the International Council for Harmonisation (ICH) guideline Q1A (R2) for stability testing of new drug substances and products. Samples of Composition 1 were packaged in moisture-protective containers and stored under two storage conditions: long-term storage at 25 °C±2 °C and 60%+5% relative humidity, and accelerated storage at 40 °C±2 °C and 75%±5% relative humidity. Beta-alanine contents were determined at day 0 (initial), 30 days, 60 days, 90 days, 120 days, 150 days, and 180 days. The results are shown in Table 7.

TABLE 7

| Storage Condition | Initial | 30 days | 60 days | 90 days | 120 days | 150 days | 180 days |
|---|---|---|---|---|---|---|---|
| Long-term | 99.97 ± | 99.88 ± | 99.86 ± | 99.82 ± | 99.75 ± | 99.64 ± | 99.57 ± |
| storage | 0.02 | 0.05 | 0.04 | 0.05 | 0.02 | 0.03 | 0.06 |
| Accelerated | 99.97 ± | 99.87 ± | 99.82 ± | 99.79 ± | 99.71 ± | 99.61 ± | 99.52 ± |
| storage | 0.02 | 0.10 | 0.06 | 0.06 | 0.03 | 0.03 | 0.07 |

The results show that the total reduction in beta-alanine content was less than 0.5% over six months under both long-term storage condition and accelerated condition, demonstrating that compositions described herein exhibited unexpectedly high stability and high resistance to moisture, which are essential properties to long-term storage and incorporation into diverse food and beverage compositions such as those described herein.

### Example 7: Stability of Composition 1 in Various Liquids

To evaluate the suitability of compositions described herein for ready-to-drink and beverage applications, an accelerated stability study was conducted where Composition 1 (600 mg) was dispersed into three liquids: (i) a protein shot containing 20 g protein (including marine collagen and whey protein) per 40 mL representing protein drinks (“protein drink”), (ii) distilled water representing aqueous beverages (“distilled water solution”), and (iii) a 1% (w/v) saline solution representing electrolyte-containing hydration beverages (“saline solution”). The resultant solutions' visual appearance, pH values, sedimentation formations, and beta-alanine contents over a storage period of 180 days were assessed.

More specifically, 600 mg of Composition 1 was dispersed in 40 mL of each liquid under controlled stirring at ambient temperature until a visually homogeneous solution was achieved. The resultant solutions were transferred into sealed containers and stored under Labtop Stability Test Chamber (model LSC-300P) at 40 °C with relativity humidity of 75%. Samples of the protein drink and distilled water solution were withdrawn at day 0, 30, 60, 90, 120, 150, and 180 for analysis. Samples of the saline solution were analyzed at day 0, 14, 28, 42, 56, 70, 84, 120, 150, and 180. At each time point, physical stability was assessed by visual inspection under standardized lighting conditions to evaluate color, turbidity, presence or absence of sedimentation, and dispersion uniformity, which are important for nutritional beverages. The pH of each solution was measured three times at ambient temperature using a calibrated digital pH meter to ensure reproducibility. Monitoring pH variation during storage is essential for identifying potential degradation related to acid-base changes. The beta-alanine content in each solution was determined using a validated LC-MS method with Waters ACQUITY UPLC H-Class system coupled with a Xevo TQ-S micro-Triple Quadrupole Mass Spectrometer (Waters Corporation, Milford, MA, USA). The results are shown in Table 8, Table 9, and Table 10 below.

TABLE 8
(distilled water solution)

| | | | | Beta Alanine |
|---|---|---|---|---|
| Day | Visual Appearance | Sedimentation | pH | Content |
| 0 | Free flow clear Light | No | 6.5 | 425 mg |
| | Brown Liquid | | | |
| 30 | Free flow clear Light | No | 6.5 | 425 mg |
| | Brown Liquid | | | |
| 60 | Free flow clear Light | No | 6.5 | 424 mg |
| | Brown Liquid | | | |
| 90 | Free flow clear Light | No | 6.5 | 424 mg |
| | Brown Liquid | | | |
| 120 | Free flow clear Light | No | 6.48 | 422 mg |
| | Brown Liquid | | | |
| 150 | Free flow clear Light | No | 6.48 | 422 mg |
| | Brown Liquid | | | |
| 180 | Free flow clear Light | No | 6.48 | 421 mg |
| | Brown Liquid | | | |

TABLE 9
(protein drink)

| | | | | Beta Alanine |
|---|---|---|---|---|
| Day | Visual Appearance | Sedimentation | pH | Content |
| 0 | Free Flow Cream Color | No | 4.80 | 426 mg |
| | Liquid | | | |
| 30 | Free Flow Cream Color | No | 4.80 | 425 mg |
| | Liquid | | | |
| 60 | Free Flow Cream color | No | 4.78 | 424 mg |
| | Liquid | | | |
| 90 | Free Flow Cream color | No | 4.78 | 424 mg |
| | Liquid | | | |
| 120 | Free Flow Cream color | No | 4.78 | 422 mg |
| | Liquid | | | |
| 150 | Free Flow Cream color | No | 4.75 | 420 mg |
| | Liquid | | | |
| 180 | Free Flow Cream color | No | 4.75 | 418 mg |
| | Liquid | | | |

TABLE 10
(saline solution)

| | | | | Beta Alanine |
|---|---|---|---|---|
| Day | Visual Appearance | Sedimentation | pH | Content |
| 0 | Light Brown Liquid | No | 5.60 | 425 mg |
| 14 | Light Brown Liquid | No | 5.60 | 425 mg |
| 28 | Light Brown Liquid | No | 5.55 | 424 mg |
| 42 | Light Brown Liquid | No | 5.55 | 424 mg |
| 56 | Light Brown Liquid | No | 5.50 | 422 mg |
| 70 | Light Brown Liquid | No | 5.50 | 422 mg |
| 84 | Light Brown Liquid | No | 5.50 | 421 mg |
| 120 | Light Brown Liquid | No | 5.50 | 421 mg |
| 150 | Light Brown Liquid | No | 5.50 | 420 mg |
| 180 | Light Brown Liquid | No | 5.50 | 420 mg |

The results show that no visual appearance changes, no sedimentation, and stable pH values over 180 days at 40 °C/75% RH, indicating that the solutions are stable. The results also show that over 180 days, the beta-alanine contents in these three solutions decreased less than 5% at 40 °C/75% RH, which meets accepted Arrhenius extrapolation criteria supporting a 24-month shelf life at ambient storage. In other words, compositions described herein can be stored in water-based beverages (e.g., soft drinks, energy drinks, and the like), electrolytic hydration beverages (e.g., sports drinks and the like), and protein drinks (including dairy-based beverages and food products) for at least 24 months.

Microbiology analyses of the distilled water solution, protein drink, and the saline solution at day 0 and day 180 were also conducted. The results show that all the test solutions at day 0 and day 180 has less than 100/g of the total microorganisms count, has less than 100/g of yeast and mold, does not contain E. coli or S. aureus, and does not contain L. monocytogenes or Salmonella (in 25 g), which are acceptable by the CFU acceptance criteria, further indicating the solutions are stable and compositions described herein can be added into beverages described herein and stored for at least 24 months.

### Example 8: Stability of Composition 2 in Protein Drink

The stability of Composition 2 in a protein drink was assessed by the procedure as described in Example 7 above. Composition 2 (a mixture of 600 mg Composition 1 and 1.5 g HMB) was dispersed in 40 mL of a protein drink containing 20 g of protein. The results are shown in Table 11 below.

TABLE 11

| | | | | Beta Alanine |
|---|---|---|---|---|
| Day | Visual Appearance | Sedimentation | pH | Content |
| 0 | Free Flow Cream Color | No | 4.50 | 420 mg |
| | Liquid | | | |
| 30 | Free Flow Cream Color | No | 4.50 | 420 mg |
| | Liquid | | | |
| 60 | Free Flow Cream color | No | 4.48 | 418 mg |
| | Liquid | | | |
| 90 | Free Flow Cream color | No | 4.48 | 418 mg |
| | Liquid | | | |
| 120 | Free Flow Cream color | No | 4.45 | 416 mg |
| | Liquid | | | |
| 150 | Free Flow Cream color | No | 4.45 | 412 mg |
| | Liquid | | | |
| 180 | Free Flow Cream color | No | 4.42 | 412 mg |
| | Liquid | | | |

The results show that no visual appearance changes, no sedimentation, and stable pH values over 180 days at 40 °C/75% RH, indicating that the solution is stable. The results also show that over 180 days, the beta-alanine content decreased less than 5% at 40 °C/75% RH, supporting a 24-month shelf life at ambient storage.

### Example 9: Compositions in a Carbonated Energy Drink

The compatibility and stability of compositions described herein were evaluated in a commercially available carbonated energy drink to assess suitability for ready-to-drink acidic beverage applications. A 250 mL serving of a standardized carbonated beverage base (initial pH~3.2 from STING brand) was used as the representative system. Composition 1 (about 560 mg-570 mg) containing about 400 mg of beta-alanine was added directly to a sealed 250 mL bottle containing a carbonated energy drink and gently agitated to ensure complete mixing while minimizing carbon dioxide loss. The bottle was immediately sealed following mixing to preserve carbonation integrity. Stability was evaluated under accelerated storage conditions (40 °C±2 °C/75±5% relative humidity) at day 0, 14, 28, 42, 56, 70, and 84 days by visual appearance, pH, and beta-alanine content. pH was measured using a calibrated digital pH meter at 25 °C. Beta-alanine content was quantified using the validated LC-MS method described above. To evaluate process robustness, thermal stability under simulated industrial hot-fill conditions was assessed by exposing filled samples to 80 °C for a defined holding period representative of commercial processing because thermal treatments are commonly used during beverage manufacturing to ensure microbial safety but may accelerate degradation of unstable ingredients. Following controlled cooling, samples were reanalyzed for visual stability and active content retention. Reference Product 1 was mixed in a separate but corresponding energy drink and used as a comparison for sensory evaluation.

It was observed that both Reference Product 1 and Composition 1 were dissolved in the energy drink instantly and completely, with no residue, sediment, turbidity, or haze formation. The addition of Reference 1 or Composition 1 to the energy drink did not change the visual appearance of the energy drink. The stability data of the energy drink containing Composition 1 is shown in Table 12.

TABLE 12

| | | | | pH | |
|---|---|---|---|---|---|
| | | | | (Compo- | Beta |
| | | Sedimen- | pH | sition 1 | Alanine |
| Day | Visual Appearance | tation | (STING) | in STING) | Content |
| 0 | Clear red solution | No | 3.2 | 3.7 | 425 mg |
| 14 | Clear red solution | No | 3.2 | 3.7 | 425 mg |
| 28 | Clear red solution | No | 3.2 | 3.7 | 424 mg |
| 42 | Clear red solution | No | 3.19 | 3.69 | 423 mg |
| 56 | Clear red solution | No | 3.19 | 3.69 | 421 mg |
| 70 | Clear red solution | No | 3.19 | 3.68 | 420 mg |
| 84 | Clear red solution | No | 3.19 | 3.68 | 420 mg |

The results show that compositions described herein are stable in carbonated energy drinks, evidenced by no visual appearance changes, no sedimentation, near zero pH changes, and near zero beta-alanine content changes, thereby demonstrating that the compositions described herein are stable in acidic beverages (e.g., soft drinks, energy drinks, and the like).

To evaluate whether users experienced paraesthesia after ingestion of the energy drink containing Composition 1, 10 panellists assessed paraesthesia intensity on a 0-10 hedonic scale (0=no sensation, 10-extreme tingling) every 14 days. Samples were blind-coded and consumed at 10 °C. As shown in Table 13, Reference Product 1 exhibited moderate tingling (with the scale of about 5.0) and Composition 1 exhibited significantly attenuated sensory intensity (with the scale of about 1), demonstrating that compositions described herein can be incorporated into acidic drinks without the user experience paraesthesia or with significantly reduced paraesthesia sensation.

TABLE 13

| Day | Reference Product 1 | Composition 1 |
|---|---|---|
| 0 | 5 | 1.0 |
| 14 | 5 | 1.0 |
| 28 | 5 | 1.0 |
| 42 | 5.2 | 1.1 |
| 56 | 5 | 1.2 |
| 70 | 5.3 | 1.0 |
| 84 | 5.0 | 1.2 |

pH values, paraesthesia sensation and beta-alanine contents of Composition 1 in the energy drink were further determined at day 120, 150, and 180. Results in Table 14 show near zero pH changes, near zero beta-alanine content changes, and near zero paraesthesia changes compared to day 84, further demonstrating stability and significant reduced paraesthesia sensation of compositions described herein in acidic drinks and thus their suitability to be incorporated into such drinks.

TABLE 14

| Day | pH | Paraesthesia sensation | Beta-Alanine Content |
|---|---|---|---|
| 120 | 3.65 | 1.2 | 418 |
| 150 | 3.65 | 1.2 | 418 |
| 180 | 3.65 | 1.2 | 418 |

In addition, Composition 1 in the energy drink also exhibited excellent thermal stability during simulated hot-fill processing, with less than 0.1% loss of beat-alanine. No off-flavors, color changes, or recrystallization were observed during post-cooling, confirming compositions described herein can withstand commercial hot-fill without impacting quality.

### Example 10: Compositions in a Dairy-Based Product

The applicability of compositions described herein in a dairy-based product was evaluated using a commercially available Greek yogurt (100 g serving; pH<5.0). Composition 1 (about 560-570 mg) containing 400 mg of beta-alanine per serving was mixed with the Greek yogurt using mechanical mixing at 1500 rpm for 10 minutes to ensure homogeneous dispersion throughout the yogurt without disrupting structural integrity and under refrigerated conditions to preserve product stability. A comparator sample containing Reference Product 1 at an identical dose was prepared in parallel. Samples were stored at 4 °C±2 °C for four weeks. Physical stability was evaluated at baseline and weekly intervals through visual inspection for phase separation, sedimentation, or structural destabilization. pH was measured at each time point using a calibrated digital pH meter. Blinded sensory evaluation was conducted by a panel (n=5) using a standardized 0-10 numerical rating scale where higher scores indicate experiencing a stronger paraesthesia sensation to assess tingling sensation, throat irritation, and mouthfeel. Assessments were performed immediately after consumption to ensure acute sensory perception.

No sedimentation, no phase separation, and no pH changes were observed in either the Reference Product 1 formulation or the Composition 1 formulation over four weeks. The mean sensory scores set forth in Table 15 demonstrate that Composition 1 in yogurt exhibited significantly reduced paraesthesia sensation compared to Reference Product 1, indicating that users consuming Composition 1 will experience significantly reduced sensations of paraesthesia when consuming dairy-based foods containing Composition 1.

TABLE 15

| Paraesthesia Sensation | Reference Product 1 | Composition 1 |
|---|---|---|
| Tingling | 4.8 | 1.2 |
| Throat Itching | 4.4 | 1.5 |
| Mouthfeel | 4.4 | 1.2 |

The present disclosure is not to be limited in terms of the particular embodiments described in this application, which are intended as illustrations of various aspects of the present disclosure. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The terminology used herein is for the purpose of describing particular embodiments only and is not intended to be limiting unless otherwise set forth herein.

1. A composition comprising:
at least one compound selected from the group consisting of beta-alanine, an ester of beta-alanine, and a salt of beta-alanine;
an anti-inflammatory agent;
an amphiphilic agent;
a stimulant; and
beta-cyclodextrin.

2. The composition of claim 1, wherein the composition comprises:
between about 10 wt. % and about 99 wt. % of the at least one compound selected from the group consisting of beta-alanine, an ester of beta-alanine, and a salt of beta-alanine;
between about 0.01 wt. % and about 50 wt. % of the anti-inflammatory agent;
between about 0.01 wt. % and about 50 wt. % of the amphiphilic agent;
between about 0.01 wt. % and about 50 wt. % of beta-cyclodextrin; and
between about 0.01 wt. % and about 50 wt. % of the stimulant.

3. The composition of claim 2, wherein the composition comprises:
between about 50 wt. % and about 80 wt. % of the at least one compound selected from the group consisting of beta-alanine, an ester of beta-alanine, and a salt of beta-alanine;
between about 0.01 wt. % and about 10 wt % of the anti-inflammatory agent;
between about 0.1 wt. % and about 10 wt. % of the amphiphilic agent;
between about 0.1 wt. % and about 20 wt. % of beta-cyclodextrin; and
between about 0.01 wt. % and about 10 wt. % of the stimulant.

4. The composition of claim 3, wherein the composition comprises:
between about 70 wt. % and about 80 wt. % of the at least one compound selected from the group consisting of beta-alanine, an ester of beta-alanine, and a salt of beta-alanine;
between about 0.1 wt. % and about 5 wt. % of the anti-inflammatory agent;
between about 1 wt. % and about 5 wt. % of the amphiphilic agent;
between about 1 wt. % and about 10 wt. % of beta-cyclodextrin; and
between about 0.1 wt. % and about 1 wt. % of the stimulant.

5. The composition of claim 1, wherein the anti-inflammatory agent is beta-caryophyllene.

6. The composition of claim 1, wherein the stimulant is caffeine.

7. The composition of claim 1, wherein the amphiphilic agent is a phospholipid.

8. The composition of claim 7, wherein the phospholipid is lecithin.

9. The composition of claim 1, wherein the composition further comprises HMB.

10. The composition of claim 1, wherein the composition is formulated as a dietary supplement.

11. The composition of claim 10, wherein the at least one compound selected from the group consisting of beta-alanine, an ester of beta-alanine, and a salt of beta-alanine is provided in an amount ranging from about 0.02 g to about 10 g per serving.

12. The composition of claim 10, wherein the at least one compound selected from the group consisting of beta-alanine, an ester of beta-alanine, and a salt of beta-alanine is provided in an amount ranging from about 0.03 g to about 6.4 g per serving.

13. The composition of claim 10, wherein the at least one compound selected from the group consisting of beta-alanine, an ester of beta-alanine, and a salt of beta-alanine is provided in an amount ranging from about 0.04 g to about 3.2 g per serving.

14. The composition of claim 10, wherein the at least one compound selected from the group consisting of beta-alanine, an ester of beta-alanine, and a salt of beta-alanine is provided in an amount ranging from about 0.05 g to about 2.24 g per serving.

15. The composition of claim 10, wherein the at least one compound selected from the group consisting of beta-alanine, an ester of beta-alanine, and a salt of beta-alanine is provided in an amount ranging from about 0.1 g to about 1 g per serving.

16. The composition of claim 10, wherein the at least one compound selected from the group consisting of beta-alanine, an ester of beta-alanine, and a salt of beta-alanine is provided in an amount ranging from about 0.2 g to about 0.8 g per serving.

17. A method for increasing athletic performance or for improving cognitive function without causing paraesthesia or with significantly reduced paraesthesia sensation in a subject, the method comprising:
administering an effective amount of a composition to the subject, wherein the composition comprises:
at least one compound selected from the group consisting of beta-alanine, an ester of beta-alanine, and a salt of beta-alanine;
an anti-inflammatory agent;
an amphiphilic agent;
a stimulant; and
beta-cyclodextrin.

18. The method of claim 17, wherein the at least one compound selected from the group consisting of beta-alanine, an ester of beta-alanine, and a salt of beta-alanine in the composition is provided in an amount ranging from 0.02 g to about 10 g per day.

19. The method of claim 17, wherein the at least one compound selected from the group consisting of beta-alanine, an ester of beta-alanine, and a salt of beta-alanine in the composition is provided in an amount ranging from 0.03 g to about 6.4 g per day.

20. The method of claim 17, wherein the at least one compound selected from the group consisting of beta-alanine, an ester of beta-alanine, and a salt of beta-alanine in the composition is provided in an amount ranging from 0.04 g to about 3.2 g per day.

21. The method of claim 17, wherein the at least one compound selected from the group consisting of beta-alanine, an ester of beta-alanine, and a salt of beta-alanine in the composition is provided in an amount ranging from about 0.05 g to about 2.24 g per day.

22. The method of claim 17, wherein the at least one compound selected from the group consisting of beta-alanine, an ester of beta-alanine, and a salt of beta-alanine in the composition is provided in an amount ranging from about 0.1 g to about 1 g per day.

23. The method of claim 17, wherein the at least one compound selected from the group consisting of beta-alanine, an ester of beta-alanine, and a salt of beta-alanine in the composition is provided in an amount ranging from about 0.2 g to about 0.8 g per day.

24. The method of claim 17, wherein the composition comprises:
between about 10 wt. % and about 99 wt. % of the at least one compound selected from the group consisting of beta-alanine, an ester of beta-alanine, and a salt of beta-alanine;
between about 0.01 wt. % and about 50 wt. % of the anti-inflammatory agent;
between about 0.01 wt. % and about 50 wt. % of the amphiphilic agent;
between about 0.01 wt. % and about 50 wt. % of beta-cyclodextrin; and
between about 0.01 wt. % and about 50 wt. % of the stimulant.

25. The method of claim 17, wherein the composition comprises:
between about 50 wt. % and about 80 wt. % of the at least one compound selected from the group consisting of beta-alanine, an ester of beta-alanine, and a salt of beta-alanine;
between about 0.01 wt. % and about 10 wt. % of the anti-inflammatory agent;
between about 0.1 wt. % and about 10 wt. % of the amphiphilic agent;
between about 0.1 wt. % and about 20 wt. % of beta-cyclodextrin; and
between about 0.01 wt. % and about 10 wt. % of the stimulant.

26. The composition of claim 17, wherein the composition comprises:
between about 70 wt. % and about 80 wt. % of the at least one compound selected from the group consisting of beta-alanine, an ester of beta-alanine, and a salt of beta-alanine;
between about 0.1 wt. % and about 5 wt. % of the anti-inflammatory agent;
between about 1 wt. % and 5 wt. % of the amphiphilic agent;
between about 1 wt. % and about 10 wt. % of beta-cyclodextrin; and
between about 0.1 wt. % and about 0.2 wt. % of the stimulant.

27. The method of claim 17, wherein the anti-inflammatory agent is beta-caryophyllene, the amphiphilic agent is a phospholipid, and the stimulant is caffeine.

28. The method of claim 17, wherein the composition further comprises HMB.

29. A method of making a beta-alanine composition, the method comprising:
step (a) adding an amphiphilic agent to water to form a first solution;
step (b) homogenizing the first solution to form a first emulsion;
step (c) adding a mixture of an anti-inflammatory agent, beta-alanine and a stimulant into the first emulsion to form a second solution;
step (d) homogenizing the second solution to form a second emulsion;
step (e) a water solution of beta cyclodextrin into the second emulsion to form a third solution;
step (f) homogenizing the third solution to form a third emulsion;
step (g) nano milling the third emulsion; and
step (h) spray-drying the third emulsion to form a dried beta-alanine composition.

30. The method of claim 29, wherein the anti-inflammatory agent is beta-caryophyllene, the amphiphilic agent is a phospholipid, and the stimulant is caffeine.
